(19) United States
(12) Patent Application Publication
(10) Pub. No.: US 2024/0150699 A1
(43) Pub. Date: May 9, 2024

(54) ELECTROPORATION PIPETTE, SYSTEM AND METHOD OF USE THEREOF

(71) Applicants: LIFE TECHNOLOGIES HOLDINGS PTE LIMITED, Singapore (SG); LIFE TECHNOLOGIES CORPORATION, Carlsbad, CA (US)

(72) Inventors: Han WEI, Singapore (SG); Chee Wai CHAN, Singapore (SG); Wui Khen LIAW, Singapore (SG); Shan Hua DONG, Singapore (SG); See Chen GOH, Singapore (SG); Huei Steven YEO, Singapore (SG); Harmon Cosme SICAT, JR., Singapore (SG); Mio Xiu Lu LING, Singapore (SG); Josh M. MEAD, Carlsbad, CA (US); Mikko MAKINEN, Carlsbad, CA (US); Beng Heng LIM, Singapore (SG); Kuan Moon BOO, Singapore (SG); Justina Linkai BONG, Singapore (SG); Chye Sin NG, Singapore (SG); Wee Liam LIM, Singapore (SG); Li Yang LIM, Singapore (SG); Way Xuang LEE, Singapore (SG)

(21) Appl. No.: 18/468,504

(22) Filed: Sep. 15, 2023

Related U.S. Application Data
(60) Provisional application No. 63/408,032, filed on Sep. 19, 2022.

(51) Int. Cl. C12M 1/42; B01L 3/02

(52) U.S. Cl. CPC C12M 35/02; B01L 3/0217; B01L 3/0275; B01L 2200/025; B01L 2200/0647; B01L 2300/0645; B01L 2300/12

(57) ABSTRACT

An electroporation system including one or more of a pipette, a pipette tip, a pipette docking assembly, and a pulse generator. The pipette docking assembly includes a pipette station, a pipette station guard, and a reservoir (e.g., a buffer tube). A method for transfecting a cell with a payload including providing an electroporation system, providing the cell, providing the payload, introducing the cell and the payload into a pipette tip, and electroporating the cell within the pipette tip by operating the electroporation system.

### CROSS REFERENCE TO RELATED APPLICATION(S)

This application claims the benefit of priority to U.S. Provisional Patent Application No. 63/408,032, filed Sep. 19, 2022, the disclosure of which is considered part of, and incorporated in its entirety by reference in the disclosure of this application.

### BACKGROUND

### Field

The present invention relates generally to cellular transfection, and more particularly to pipettes, pipette tips, assemblies, electroporation systems, as well methods for transfecting a cell.

### Background Information

Some electroporation systems include a pipette for holding the target cells and the payload (e.g., nucleic acid and/or proteins to be introduced into the target cells) and an electrical pulse generator for providing an electrical pulse to the target cells. The pipette can be connected to or inserted into a docking station associated with the electrical pulse generator to enable the electrical pulse generated by the electrical pulse generator to reach the target cells.

For example, a pipette electrode in conductive communication with one end of the pipette chamber of the pipette (e.g., holding the target cells and payload) can interface with a first electrode on the docking station. The tip of the pipette (e.g., including the open end of the pipette chamber) can be inserted into a buffer solution (e.g., within a reservoir) that is in conductive communication with a second electrode on the docking station, thereby exposing the open end of the pipette chamber to the buffer solution. With the pipette so connected to the docking station, the electrical pulse generator can provide an electrical pulse to the first and second electrodes of the docking station, thereby allowing the electrical pulse to travel through the pipette chamber to reach and electroporate the target cells.

Existing electroporation systems, as well as system components (e.g., pipettes, pipette tips, pipette docking assemblies and pulse generators) suffer from a number of shortcomings and there is an ongoing need and desire for improved electroporation systems including improved components.

### SUMMARY

Various aspects the present disclosure extend at least to electroporation systems, components thereof, and/or methods associated therewith.

In one aspect, the present disclosure provides an electroporation system. In embodiments, the system includes one or more of a pipette, a pipette tip, a pipette docking assembly, and a pulse generator. In some embodiments, the pipette docking assembly includes a pipette station, a pipette station guard, and a reservoir.

In another aspect, the present disclosure provides a pipette. In embodiments, the pipette includes a proximal section having a handle, a distal section configured to reversibly attach to a pipette tip, a first actuator disposed in the proximal section that when actuated is operable to control: i) a pipetting function of the pipette; and ii) grasping and ungrasping of a plunger disposed within a lumen of the pipette tip, and a second actuator disposed in the proximal section that when actuated is operable to cause the pipette tip to detach from the distal section of the pipette. In various embodiments, the pipette includes a pipette electrode disposed in the distal section which is electrically coupled to the plunger when the plunger is operably coupled to the first actuator.

In yet another aspect, the present disclosure provides a pipette tip configured to reversibly attach to a pipette. In embodiments, the pipette tip includes a tip sleeve defining a lumen extending from a proximal end of the pipette tip to a distal end of the pipette tip, a plunger at least partially disposed within the lumen, the plunger being composed of an electrically conductive material and configured to translate along the lumen to facilitate aspirating fluid into, and/or dispensing fluid from, the lumen, and an attachment interface disposed at the proximal end of the pipette tip, the attachment interface comprising one or more tabs configured to engage with the pipette. In some embodiments, the attachment interface includes one or more tabs configured to engage with a retention platform of a distal section of a pipette. In some embodiments, the one or more tabs are configured to interact with a biasing member of the pipette during attachment of the pipette tip with the pipette via the retention platform. In various embodiments, the pipette tip has a sample volume capacity of between 10 μL and 100 μL.

In various aspects, the disclosure provides pipette assemblies including a pipette reversibly attached to a pipette tip. In some embodiments, the pipette of the assembly includes a proximal section having a handle, a distal section having a tip interface, and a first actuator disposed in the proximal section. The pipette tip reversibly attached to the pipette of the assembly includes a tip sleeve defining a lumen extending from a proximal end of the pipette tip to a distal end of the pipette tip, a plunger at least partially disposed within the lumen, and an attachment interface disposed at the proximal end of the pipette tip. In various embodiments, the plunger is reversibly operably coupled to the first actuator, and when operably coupled, performs a pipetting function upon actuation of the first actuator by translating along the lumen. Additionally, the tip sleeve is reversibly attached to the distal section via one or more tabs of the attachment interface engaged with a retention platform of the distal section. In embodiments, the pipette of the assembly further includes a pipette electrode disposed in the distal section and electrically coupled to the plunger when the plunger is operably coupled to the first actuator.

In some embodiments, the pipette of the assembly includes a proximal section having a handle, a distal section having a tip interface, a first actuator disposed in the proximal section, and a gripper mechanism disposed in the distal section having a gripper jaw. The pipette tip reversibly attached to the pipette of the assembly includes a tip sleeve defining a lumen extending from a proximal end of the pipette tip to a distal end of the pipette tip, and a plunger at least partially disposed within the lumen. In various embodiments, the plunger is reversibly operably coupled to the first actuator via the gripper jaw, and when operably coupled, performs a pipetting function upon actuation of the first actuator by translating along the lumen. In embodiments, the pipette of the assembly further includes a pipette electrode disposed in the distal section and electrically coupled to the plunger when the plunger is operably coupled to (e.g., grasped by) the gripper jaw.

In another aspect, the disclosure provides a pulse generator that includes one or more connection ports. Each particular connection port of the one or more connection ports includes a respective port door. Each respective port door includes a respective biasing element for biasing the respective port door into a closed configuration for preventing access to the particular connection port. In some instances, each respective port door includes a respective tool interface configured to receive a port door tool. The port door tool is configured to interact with the respective tool interface to counteract the respective biasing element of the respective port door to bring the respective port door into an open configuration to provide access to the particular connection port. Insertion of a port connection component into the particular connection port can maintain the counteracting of the respective biasing element to maintain the respective port door in the open configuration, whereas disconnection of the port connection component from the particular connection port can remove the counteracting of the respective biasing element to allow the respective port door to return to the closed configuration.

In still another aspect, the present disclosure provides a method for transfecting a cell with a payload. The method includes providing an electroporation system of the disclosure, providing the cell, providing the payload, introducing the cell and the payload into a pipette tip attached to a pipette of the system, and electroporating the cell by operating the electroporation system. In some embodiments, the cell is a mammalian cell. In some embodiments, the payload includes a nucleic acid, a protein, or a combination thereof.

Additional features and advantages will be set forth in the description which follows, and in part will be obvious from the description, or may be learned by the practice of the teachings herein. Features and advantages of the systems described herein may be realized and obtained by means of the instruments and combinations particularly pointed out in the appended claims. Features of the present invention will become more fully apparent from the following description and appended claims or may be learned by the practice of the invention as set forth hereinafter.

### BRIEF DESCRIPTION OF THE DRAWINGS

For a fuller understanding of the nature and advantages of the present invention, reference should be made to the following detailed description taken in connection with the accompanying drawings. It is appreciated that these drawings depict only typical embodiments of the invention and are therefore not to be considered limiting of its scope. The disclosure will be described and explained with additional specificity and detail through the use of the accompanying drawings.

FIG. 1 is a schematic diagram illustrating example components of an electroporation system of the disclosure, as well as components thereof in one embodiment of the disclosure.

FIGS. 2A-2B are schematic diagrams illustrating aspects of example pipette tips (e.g., consumable pipette tips) for use with the electroporation system in embodiments of the disclosure. FIG. 2A is a schematic diagram illustrating aspects of an example pipette tip in one embodiment of the disclosure. FIG. 2B is a schematic diagram illustrating aspects of an example pipette tip in one embodiment of the disclosure.

FIGS. 3A-3B are schematic diagrams showing exploded views of the pipette tips depicted in FIGS. 2A-2B. FIG. 3A is an exploded view of the pipette tip depicted in FIG. 2A. FIG. 3B is an exploded view of the pipette tip depicted in FIG. 2B.

FIGS. 4A-4B are schematic diagrams illustrating aspects of plungers of pipette tips in embodiments of the disclosure. FIG. 4A is an exploded view of an example plunger. FIG. 4B is a schematic diagram of the plunger depicted in FIG. 4A as assembled.

FIGS. 5A-5B are schematic diagrams illustrating aspects of a pipette tip in embodiments of the disclosure. FIG. 5A is a cross sectional view of a pipette tip in one embodiment of the disclosure. FIG. 5B is an expanded cross sectional view of the distal portion of the pipette tip depicted in FIG. 5A.

FIG. 6 a schematic diagram illustrating aspects of a plunger of a pipette tip in embodiments of the disclosure.

FIGS. 7A-7B are schematic diagrams illustrating a pipette tip in one embodiment of the disclosure. FIG. 7A is a schematic diagram of a pipette tip of the disclosure. FIG. 7B is an expanded view of the distal portion of the pipette tip depicted in FIG. 7A.

FIGS. 8A-8B are schematic diagrams illustrating components of a pipette and pipette tip in embodiments of the disclosure. FIG. 8A is a perspective view of a pipette in one embodiment of the disclosure. FIG. 8B is a perspective view of a pipette tip for use with the pipette depicted in FIG. 8A in one embodiment of the disclosure.

FIGS. 9A-9E are schematic diagrams illustrating the interaction between a pipette and pipette tip in embodiments of the disclosure. FIG. 9A is a schematic diagram illustrating the interaction of an attachment interface of the pipette tip with an engagement interface of the pipette. FIG. 9B is a schematic diagram illustrating the interaction of an attachment interface of the pipette tip with an engagement interface of the pipette. FIG. 9C is a cross sectional view of the distal end of the pipette coming into engagement with the proximal end of the pipette tip.

FIG. 9D is a cross sectional view of the distal end of the pipette coming into engagement with the proximal end of the pipette tip. FIG. 9E is a cross sectional view of the tip interface of the distal end of the pipette attached to the attachment interface of the proximal end of the pipette tip.

FIGS. 10A-10B are cross sectional views illustrating the pipetting functions of a pipette and attached pipette tip in embodiments of the disclosure. FIG. 10A is a cross sectional view illustrating transition of components of the pipette and the pipette tip for aspirating fluid into the lumen of the pipette tip. FIG. 10B is a cross sectional view illustrating transition of components of the pipette and the pipette tip for dispensing fluid from the lumen of the pipette tip.

FIGS. 11A-11B are schematic diagrams illustrating functional and structural aspects of a pipette and pipette tip in embodiments of the disclosure. FIG. 11A is a schematic diagram illustrating operation of the pipette. FIG. 11B is a schematic diagram illustrating operation of the pipette.

FIG. 12 is a perspective view illustrating aspects of an example pipette station guard of an electroporation system in embodiments of the disclosure.

FIGS. 13A-13B are perspective views illustrating assembly of the pipette station guard depicted in FIG. 12 with a pipette station in embodiments of the disclosure. FIG. 13A shows insertion and rotation of the pipette station guard during assembly with the pipette station.

FIG. 13B shows the pipette station guard assembled with the pipette station in which the base of the pipette station guard is pushed toward the pipette station to complete assembly.

FIG. 14 illustrates an example reservoir, also referred to as a buffer tube, of an electroporation system in embodiments of the disclosure.

FIGS. 15A-15B are perspective views illustrating assembly of the reservoir depicted in FIG. 14 with the pipette station guard and pipette station depicted in FIG. 13B in embodiments of the disclosure. FIG. 15A illustrates alignment of the reservoir with the pipetted station guard for assembly with the assembled pipette station and pipetted station guard.

FIG. 15B shows the reservoir assembled with the pipette station and pipetted station guard to form a fully assembled pipette docking assembly.

FIG. 16 is a cross sectional side view of a fully assembled pipette docking assembly in embodiments of the disclosure.

FIG. 17 is a cross sectional side view of a fully assembled pipette docking assembly in embodiments of the disclosure.

FIGS. 18A-18B are perspective views illustrating assembly of a pipette into the reservoir of the pipette docking assembly depicted in FIG. 15B to perform an electroporation procedure. FIG. 18A illustrates alignment of the pipette with the pipetted station guard for assembly with the pipette docking assembly. FIG. 18B shows the pipette assembled with the pipette docking assembly.

FIG. 19 is a side view illustrating aspects of assembly of a pipette with a pipette docking assembly in embodiments of the disclosure.

FIG. 20 is a rear elevation view of a pulse generator illustrating aspects of example port doors in embodiments of the disclosure.

FIG. 21 is a perspective view illustrating aspects of components of a pulse generator in embodiments of the disclosure.

FIG. 22 is an expanded, sectional, perspective view illustrating aspects of components of a pulse generator in embodiments of the disclosure.

FIG. 23 is an expanded, sectional, perspective view illustrating aspects of components of a pulse generator in embodiments of the disclosure.

FIGS. 24A-24B are expanded, sectional, perspective views illustrating aspects of components of a pulse generator in embodiments of the disclosure. FIG. 24A shows a connection component for receiving a portable drive (e.g., USB) device. FIG. 24B shows a USB device inserted into the connection component.

FIG. 25A-25B are expanded perspective views illustrating aspects of components of a pulse generator in embodiments of the disclosure. FIG. 25A shows a USB device inserted into a connection component. FIG. 25B shows a door covering the connection component which automatically closes upon removal of a connected device (e.g., USB device).

FIGS. 26A-26B are perspective views illustrating aspects of an example cable adapter. FIG. 26A shows features of the cable adapter. FIG. 26B is an exploded view of components of the cable adapter.

FIGS. 27A-27C are sectional views illustrating aspects of an example cable adapter and pulse generator. FIG. 27A shows a sectional view of the cable adapter and a pulse generator. FIG. 27B shows a section view of the pulse generator and attachment of the cable adapter with corresponding attachment features aligned. FIG. 27C shows a section view of attachment of the cable adapter to the pulse generator by sliding the cable adapter downward with respect to the pulse generator once a mounting hole of the pulse generator and a mounting feature of the cable adapter are aligned as in FIG. 27B.

FIGS. 28A-28C illustrate aspects of an example cable binding assembly of an electroporation system. FIG. 28A shows a cable binding assembly with connection portions for electrically coupling to a pulse generator and pipette station. FIG. 28B shows features of the connection portion of the cable binding assembly configured to electronically couple to the pulse generator. FIG. 28C shows features of the connection portion of the cable binding assembly configured to electronically couple to the pipette station.

FIGS. 29A-29B are schematic diagrams illustrating aspects of a clip of the cable assembly. FIG. 29A shows features of the clip for engaging cables. FIG. 29B shows structural features of the assembled clip of FIG. 29A.

FIG. 30 is a cross sectional schematic diagram of a pipette docking assembly with a pipette and attached pipette tip including a sample docked within the docking assembly for conducting an electroporation procedure.

FIG. 31 is a graphical representation illustrating aspects of an example electrical pulse generated by a pulse generator of an electroporation system in an embodiment of the disclosure.

FIG. 32 is a graphical representation illustrating aspects of an example electrical pulse generated by a pulse generator of an electroporation system in an embodiment of the disclosure.

FIG. 33 is a schematic diagram showing example circuitry architecture that may be implemented in a pulse generator of an electroporation system in embodiments of the disclosure.

FIG. 34 is a schematic diagram showing example circuitry architecture that may be implemented in a pulse generator of an electroporation system in embodiments of the disclosure.

FIG. 35 is a graphical plot illustrating example electrical waveforms generated by a pulse generator in embodiments of the disclosure.

FIG. 36 is a graphical plot illustrating example electrical waveforms generated by a pulse generator in embodiments of the disclosure.

FIG. 37 is a graphical plot illustrating example electrical waveforms generated by a pulse generator in embodiments of the disclosure.

FIG. 38 is a graphical plot illustrating aspects of arcing and arc detection associated with electroporation systems.

FIG. 39 is a graphical plot illustrating aspects of arcing and arc detection associated with electroporation systems.

FIG. 40 is a schematic diagram illustrating aspects of circuitry of a pulse generator in embodiments of the disclosure.

FIG. 41 is a schematic diagram illustrating aspects of circuitry of a pulse generator in embodiments of the disclosure.

FIG. 42 is a schematic diagram illustrating aspects of circuitry of a pulse generator in embodiments of the disclosure.

FIG. 43 is a graphical plot illustrating aspects of arcing and arc detection associated with electroporation systems.

FIG. 44 is a graphical plot illustrating aspects of arcing and arc detection associated with electroporation systems.

FIG. 45 is a graphical plot illustrating aspects of arcing and arc detection associated with electroporation systems.

FIG. 46 is a graphical plot illustrating aspects of arcing and arc detection associated with electroporation systems.

FIG. 47 is a front, right perspective view of one embodiment of an electroporation system of the disclosure.

FIG. 48 is a front, right, perspective view of one embodiment of a pulse generator of the disclosure.

FIG. 49 is a front elevation view of the pulse generator depicted in FIG. 48.

FIG. 50 is a rear elevation view of the pulse generator depicted in FIG. 48.

FIG. 51 is a right side elevation view of the pulse generator depicted in FIG. 48.

FIG. 52 is a left side elevation view of the pulse generator depicted in FIG. 48.

FIG. 53 is a top plan view of the pulse generator depicted in FIG. 48.

FIG. 54 is a bottom plan view of the pulse generator depicted in FIG. 48.

FIG. 55 is a front, right, perspective view of one embodiment of a docking station assembled with a pipette station guard.

FIG. 56 is a front elevation view of the docking station and pipette station guard assembly depicted in FIG. 55.

FIG. 57 is a rear elevation view of the docking station and pipette station guard assembly depicted in FIG. 55.

FIG. 58 is a right side elevation view of the docking station and pipette station guard assembly depicted in FIG. 55.

FIG. 59 is a left side elevation view of the docking station and pipette station guard assembly depicted in FIG. 55.

FIG. 60 is a top plan view of the docking station and pipette station guard assembly depicted in FIG. 55.

FIG. 61 is a bottom plan view of the docking station and pipette station guard assembly depicted in FIG. 55.

FIG. 62 is a front, right, perspective view of one embodiment of a pipette station assembled with a pipette station guard and a reservoir.

FIG. 63 is a front elevation view of the pipette station, pipette station guard and reservoir assembly depicted in FIG. 62.

FIG. 64 is a rear elevation view of the pipette station, pipette station guard and reservoir assembly depicted in FIG. 62.

FIG. 65 is a right side elevation view of the pipette station, pipette station guard and reservoir assembly depicted in FIG. 62.

FIG. 66 is a left side elevation view of the pipette station, pipette station guard and reservoir assembly depicted in FIG. 62.

FIG. 67 is a top plan view of the pipette station, pipette station guard and reservoir assembly depicted in FIG. 62.

FIG. 68 is a bottom plan view of the pipette station, pipette station guard and reservoir assembly depicted in FIG. 62.

FIG. 69 is a front, right, perspective view of one embodiment of a reservoir.

FIG. 70 is a front elevation view of the reservoir depicted in FIG. 69.

FIG. 71 is a rear elevation view of the reservoir depicted in FIG. 69.

FIG. 72 is a right side elevation view of the reservoir depicted in FIG. 69.

FIG. 73 is a left side elevation view of the reservoir depicted in FIG. 69.

FIG. 74 is a top plan view of the reservoir depicted in FIG. 69.

FIG. 75 is a bottom plan view of the reservoir depicted in FIG. 69.

FIG. 76 is a front, right, perspective view of one embodiment of a pipette.

FIG. 77 is a front elevation view of the pipette depicted in FIG. 76.

FIG. 78 is a rear elevation view of the pipette depicted in FIG. 76.

FIG. 79 is a right side elevation view of the pipette depicted in FIG. 76.

FIG. 80 is a left side elevation view of the pipette depicted in FIG. 76.

FIG. 81 is a top plan view of the pipette depicted in FIG. 76.

FIG. 82 is a bottom plan view of the pipette depicted in FIG. 76.

FIG. 83 is a front, right, perspective view of one embodiment of a pipette tip having a sample volume capacity of 100 μL.

FIG. 84 is a front elevation view of the pipette tip depicted in FIG. 83.

FIG. 85 is a rear elevation view of the pipette tip depicted in FIG. 83.

FIG. 86 is a right side elevation view of the pipette tip depicted in FIG. 83.

FIG. 87 is a left side elevation view of the pipette tip depicted in FIG. 83.

FIG. 88 is a top plan view of the pipette tip depicted in FIG. 83.

FIG. 89 is a bottom plan view of the pipette tip depicted in FIG. 83.

FIG. 90 is a front, right, perspective view of one embodiment of a pipette tip having a sample volume capacity of 10 μL.

FIG. 91 is a front elevation view of the pipette tip depicted in FIG. 90.

FIG. 92 is a rear elevation view of the pipette tip depicted in FIG. 90.

FIG. 93 is a right side elevation view of the pipette tip depicted in FIG. 90.

FIG. 94 is a left side elevation view of the pipette tip depicted in FIG. 90.

FIG. 95 is a top plan view of the pipette tip depicted in FIG. 90.

FIG. 96 is a bottom plan view of the pipette tip depicted in FIG. 90.

### DETAILED DESCRIPTION

Before describing various embodiments of the present disclosure in detail, it is to be understood that this disclosure is not limited to the parameters of the particularly exemplified systems, methods, apparatus, assemblies, products, processes, consumables, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the terminology used herein is for the purpose of describing the embodiments and is not necessarily intended to limit the scope of the claimed invention.

Furthermore, it is understood that for any given component or embodiment described herein, any of the possible candidates or alternatives listed for that component may generally be used individually or in combination with one another, unless implicitly or explicitly understood or stated otherwise. Additionally, it will be understood that any list of such candidates or alternatives is merely illustrative, not limiting, unless implicitly or explicitly understood or stated otherwise.

In addition, unless otherwise indicated, numbers expressing quantities, constituents, distances, or other measurements used in the specification and claims are to be understood as being modified by the term “about,” as that term is defined herein. The terms “about” and “approximate”, as used herein when referring to a measurable value such as an amount, dose, time, temperature, activity, level, number, frequency, percentage, dimension, size, amount, weight, position, length and the like, is meant to encompass variations of ±15%, ±10%, ±5%, ±1%, ±0.5%, or even±0.1% of the specified amount, dose, time, temperature, activity, level, number, frequency, percentage, dimension, size, amount, weight, position, length and the like.

The term “comprising” which is synonymous with “including,” “containing,” “having” or “characterized by,” is inclusive or open-ended and does not exclude additional, unrecited elements or method steps.

It will be noted that, as used in this specification and the appended claims, the singular forms “a,” “an” and “the” include plural referents unless the content clearly dictates otherwise. Thus, for example, reference to an “inlet” includes one, two, or more inlets.

As used in the specification and appended claims, directional terms, such as “top,” “bottom,” “left,” “right,” “up,” “down,” “upper,” “lower,” “inner,” “outer,” “internal,” “external,” “interior,” “exterior,” “proximal,” “distal” and the like are used herein solely to indicate relative directions and are not otherwise intended to limit the scope of the disclosure or claims.

Where possible, like numbering of elements have been used in various figures. Furthermore, alternative configurations of a particular element may each include separate letters appended to the element number. Accordingly, an appended letter can be used to designate an alternative design, structure, function, implementation, and/or embodiment of an element or feature without an appended letter. For instance, an element “80” may be embodied in an alternative configuration and designated “80a.” Similarly, multiple instances of an element and or sub-elements of a parent element may each include separate letters appended to the element number. In each case, the element label may be used without an appended letter to generally refer to all instances of the element or any one of the alternative elements. Element labels including an appended letter can be used to refer to a specific instance of the element or to distinguish or draw attention to multiple uses of the element.

Various aspects of the present devices, systems, and methods may be illustrated with reference to one or more exemplary embodiments. As used herein, the term “embodiment” means “serving as an example, instance, or illustration,” and should not necessarily be construed as preferred or advantageous over other embodiments disclosed herein.

Various aspects of the present devices and systems may be illustrated by describing components that are coupled, attached, and/or joined together. As used herein, the terms “coupled”, “attached”, “connected” and/or “joined” are used to indicate either a direct connection between two components or, where appropriate, an indirect connection to one another through intervening or intermediate components. In contrast, when a component is referred to as being “directly coupled”, “directly attached”, “directly connected” and/or “directly joined” to another component, there are no intervening elements present.

Unless defined otherwise, all technical and scientific terms used herein have the same meaning as commonly understood by one of ordinary skill in the art to which the present disclosure pertains. Although a number of methods and materials similar or equivalent to those described herein can be used in the practice of the present disclosure, the preferred materials and methods are described herein.

Implementations of the present disclosure extend at least to pipettes (e.g., multichannel pipettes) used for electroporation, as well as electroporation systems and/or components thereof which utilize such pipettes. The disclosed aspects and embodiments may be implemented to address various shortcomings associated with at least some conventional pipettes and electroporation systems and/or techniques. The following discussion outlines some example improvements and/or practical applications that may be provided by the disclosed embodiments. It will be appreciated, however, that the following are examples only and that the embodiments described herein are in no way limited to the example improvements discussed herein.

Some implementations of the present disclosure provide pipettes that are designed to simplify pipetting operations required for processing of samples, as well as reduce muscle strain of a pipette user associated with use of the pipette to electroporate samples. The unique design of the pipettes described herein reduces muscular stress and/or fatigue of a user by reducing the forces involved with manually performing pipetting functions to repeatedly process samples. Additionally, the pipettes of the present disclosure are designed to utilize pipette tips in which a clip-on connection between pipette tips and pipettes is achieved. Use of clip-on connections combined with pipette design improvements, as well as other components of the electroporation system increases sample processing efficiency and reliability, as well as ease of use of the electroporation system.

Some existing electroporation pipettes have pipette tips with electrically conductive plunger components for carrying electrical current during electroporation. However, such devices can exhibit high frictional force caused by interference between the plunger and the chamber (or lumen) wall of the pipette tip, which can affect the functioning of the pipette and/or cause retardation during dispensing and/or aspiration.

At least some implementations of the present disclosure provide an electroporation pipette tip with a plunger that includes a sealing component for reducing the contact area between the plunger and the chamber (or lumen) wall of the pipette tip. The sealing component can take on various forms, such as polymer sleeve and/or O-ring. Such features may reduce the frictional force between the plunger and the chamber (or lumen) wall, thereby facilitating improved pipetting functionality that is less susceptible to performance degradation over time.

Conventional consumable pipette tips typically connect to conventional electroporation pipettes via an interference fit. To mount conventional pipette tips to conventional electroporation pipettes, users often are required to exert significant downward force to press the electroporation pipette into the pipette tip while maintaining force on the pipette plunger trigger (e.g., actuator) to enable a plunger gripper of the electroporation pipette to grip the plunger of the pipette tip. This can result in user fatigue and/or frustration. Furthermore, conventional pipette tips of conventional electroporation pipettes often require application of a large amount of force on an ejection button (e.g., about 60 N) to allow pipette tip ejection, which can further contribute to user fatigue and/or frustration.

At least some implementations of the present disclosure provide pipette assemblies that enable a clip-on connection between pipette tips and pipettes (e.g., via one or more tabs of the pipette tip clipping onto a retention platform of the pipette). Such features allow for a two-part pipette tip attachment process, where the user presses the pipette into the pipette tip to facilitate a clip-on connection with a tip sleeve of the pipette tip and subsequently, or simultaneously, actuates an actuator (e.g., a trigger, button or the like) by depressing the actuator to cause a gripper jaw of the pipette to reversibly grasp/grip the plunger of the pipette tip. Such functionality can provide users with a more convenient pipette tip loading process that results in less muscular stress and/or fatigue. In various embodiments, users can optionally actuate a first actuator (e.g., depress a plunger trigger) while pressing the pipette into the pipette tip to perform tip loading. Similarly, the clip-on connection between the pipette tip and the pipette can allow for a two-part pipette tip ejection process, where the user first depresses a second actuator (e.g., ejection button) to eject the tip sleeve (e.g., outer portion of the pipette tip) without ejecting the plunger and subsequently depresses the first actuator of the pipette to release the plunger from the gripper jaw of the pipette tip. Such functionality enables a reduced peak ejection force for facilitating tip detachment/ejection and can thereby reducing muscular stress and/or fatigue.

In many existing electroporation systems, the reservoir that holds the buffer solution (e.g., a buffer tube or other reservoir) is easily removed from the pipette docking assembly, to prevent inadvertent removal of the reservoir from the assembly when withdrawing the pipette tip from the reservoir of the assembly. Such inadvertent removal can result in spillage and/or damage to pipette tips. In some embodiments, the reservoir (e.g., buffer tube) is held by a pipette station guard assembled with the pipette station for protecting users against electrical shock. In contrast, conventional station guards are easily inadvertently removed during withdrawal of a pipette from a reservoir, or even during electroporation, which presents an electrical shock hazard.

At least some implementations of the present disclosure provide a pipette station guard that locks into the docking station (e.g., “pipette station”) via movement of the station guard in a locking direction that is different from the pipette removal direction for removing the pipette from the reservoir. The reservoir inserts into an opening of the station guard and locks to the station guard. The reservoir can be released from the station guard by application of force (e.g., on latching members) in a force application direction that is different from the pipette removal direction for removing the pipette from the reservoir. Such features reduce or eliminate the incidence of inadvertent removal of reservoirs (e.g., buffer tubes) and/or station guards from pipette stations during pipette removal, thereby reducing or avoiding spillage and/or pipette tip damage.

In many existing electroporation systems, the high-voltage cables connecting the pipette station to the electrical pulse generator are integrally formed with the pipette station, often necessitating replacement of the entire pipette station when cable fault occurs (e.g., due to aging and/or cable insulation degradation).

In contrast, the present disclosure provides a pulse generator with external high-voltage cable connection ports (and, in at least some instances, low-voltage cable connection ports) to enable the high-voltage cables to be independent from the pulse generator, thereby enabling replacement of individual cables when cable fault occurs (as opposed to necessitating replacement of the entire pulse generator in response to cable fault). At least some implementations of the present disclosure may further provide a cable adapter that is selectively mountable to the pulse generator, thereby facilitating improved cable management functionality (e.g., to consolidate cables during storage and/or manage cables connected to multiple pipette stations). Furthermore, at least some implementations of the present disclosure may provide high-voltage cables that are coupled to low-voltage cables via a braid and clip assembly that further promotes convenient cable management.

In many existing electroporation systems, the pulse generator includes separate high-voltage and low-voltage power supplies for facilitating electroporation (via the high-voltage power supply) and other incidental functions such as data transmission (via the low-voltage power supply). In the event that isolation between the high-voltage and low-voltage circuits fails, high-voltage discharge may occur through contact with low-voltage components (e.g., connection ports such as USB ports, LAN ports, Wi-Fi dongle ports, and the like). At least some conventional electroporation systems included port covers on low-voltage components to prevent user harm from high-voltage discharge through low-voltage components. However, conventional port covers often require manual removal of the port cover to enable connection of external components to the underlying port and often require manual replacement of the port cover after disconnection of the external components from the underlying port. Users often forget to re-connect the port cover after disconnection of external components, which can expose users to a risk of injury and/or death from high-voltage discharge through low-voltage connection ports.

At least some implementations of the present disclosure provide a low-voltage port door system that enables automatic closing of port doors after disconnection of external components from the ports associated with the port doors. For example, each individual port door may include a biasing member that constantly biases the port door toward a closed position, thereby automatically forcing the port door into the closed position after removal of interfering objects (e.g., plugs of external components). Such functionality may reduce user exposure to risks associated with high-voltage discharge through low-voltage connection ports. The port door system may still require manual user action to open port doors for initial connection of external components to ports. Such port door systems would comply with various safety compliance standards (e.g., IEC61010-1:2010/AMD1:2016).

Many conventional electroporation systems include a pulse generator that uses a charging circuit to charge a large capacitor to the target voltage and then uses a high-speed, high-voltage electronic switch that connects the capacitor to the pipette station to deliver the high-voltage pulse to the target cells (e.g., within the pipette chamber connected to the docking station). In at least some circumstances, such as when the load resistance is small (e.g., for larger pipette tip sizes), the voltage applied to the target cells can droop through the duration of the pulse. A droop in voltage can adversely affect electroporation results.

At least some implementations of the present disclosure provide a pulse generator that utilizes feedback loop control in which capacitors are charged to a voltage above the target voltage and, during discharge, the voltage is regulated to produce a steady supply in accordance with the target voltage (and/or pulse width and/or waveform settings). Such functionality can improve the consistency and predictability of electroporation results.

During high-voltage electroporation (e.g., 500V to 2,500V), arcing may occur in response to bubbles and/or other contaminants in the chamber/lumen of the pipette tip. Arcing can cause poor electroporation results and/or failure of electroporation. Conventional electroporation systems fail to include systems for detecting arcing during electroporation. Thus, to determine whether arcing occurs, users typically rely on real-time visual monitoring of target cells to detect whether a spark is observed during electroporation. However, because such sparks occur in the millisecond time range, users can often fail to detect a visible spark during electroporation. Furthermore, in some instances, arcing can occur in the absence of a visible spark (e.g., when the electroporation voltage is relatively low).

At least some implementations of the present disclosure include an arcing detection module for automatically detecting sudden drops in electroporation pulse current, which are indicative of arcing. The arcing detection module can include an amplifier, band pass filter, and comparator to detect whether a falling current signal is present under various current profiles (e.g., for different types/sizes of pipette tips, for different buffer solutions, etc.). When arcing is detected, a notification or other indication that arcing has occurred may be provided to users. Users may thus become aware of whether arcing has occurred during electroporation without relying on human monitoring during electroporation, which can help users properly interpret electroporation results.

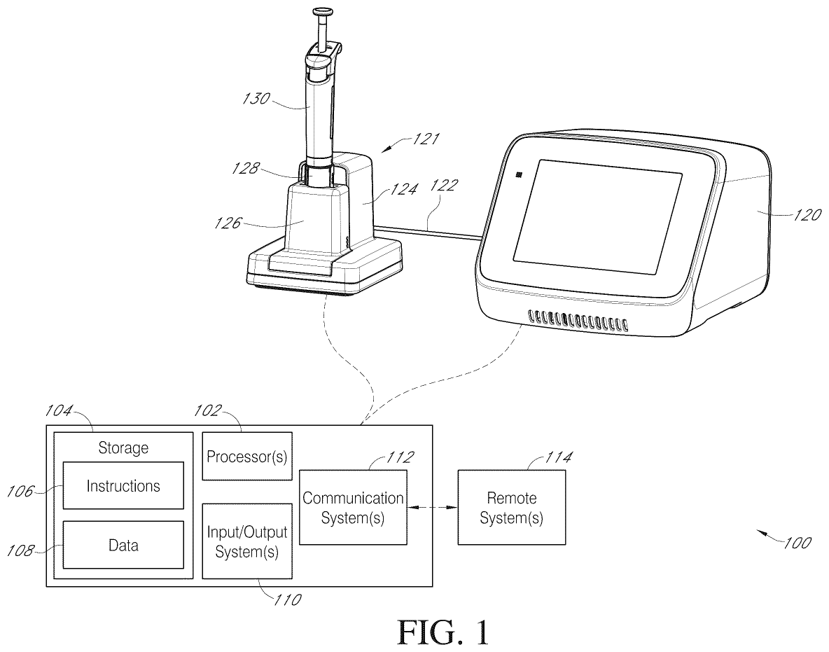

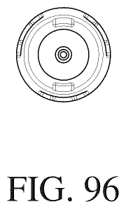

Attention will now be directed to FIGS. 1 through 96 which provide various supporting illustrations related to the disclosed embodiments as described in detail herein.

Electroporation System

FIG. 1 illustrates various example components of an electroporation system 100 that may be used to implement one or more disclosed embodiments. For example, the electroporation system 100 of FIG. 1 may be configured to facilitate cellular transfection by applying a current to target cells to introduce a payload into the target cells to facilitate, for example, production of genetically modified cells which may be used to a cellular therapy product. Although FIG. 1 illustrates an electroporation system 100 as including particular components, one will appreciate in view of the present disclosure, that an electroporation system 100 may include any number of additional and/or alternative components. Furthermore, one will appreciate, in view of the present disclosure, that the principles disclosed herein are not limited to the particular form and/or features of the electroporation system 100, or particular components thereof, shown in FIG. 1.

FIG. 1 illustrates that an electroporation system 100 may include processor(s) 102, storage 104, input/output system(s) 110 (I/O system(s) 110), and communication system(s) 112. The processor(s) 102 may comprise one or more sets of electronic circuitries that include any number of logic units, registers, and/or control units to facilitate the execution of computer-readable instructions (e.g., instructions that form a computer program). Such computer-readable instructions may be stored within storage 104. The storage 104 may comprise physical system memory and may be volatile, non-volatile, or some combination thereof. Furthermore, storage 104 may comprise local storage, remote storage (e.g., accessible via communication system(s) 112 or otherwise), or some combination thereof. Additional details related to processors (e.g., processor(s) 102), computer storage media (e.g., storage 104), and other computer components will be provided hereinafter.

The processor(s) 102 may be configured to execute instructions 106 stored within storage 104 to perform certain actions and/or commands (e.g., voltage/current control, user interface presentation, receiving user input, component detection, etc.). The actions may rely at least in part on data 108 stored on storage 104 in a volatile or non-volatile manner.

In some instances, the actions may rely at least in part on communication system(s) 112 for receiving data from remote system(s) 114, which may include, for example, computing devices, sensors, and/or others. The communications system(s) 112 may comprise any combination of software or hardware components that are operable to facilitate communication between on-system components/devices and/or with off-system components/devices. For example, the communications system(s) 112 may comprise ports, buses, or other physical connection apparatuses for communicating with other devices/components. Additionally, or alternatively, the communications system(s) 112 may comprise systems/components operable to communicate wirelessly with external systems and/or devices through any suitable communication channel(s), such as, by way of non-limiting example, Bluetooth, ultra-wideband, WLAN, infrared communication, and/or others.

Furthermore, FIG. 1 illustrates that an electroporation system 100 may comprise or be in communication with I/O system(s) 110. I/O system(s) 110 may include any type of input or output device such as, by way of non-limiting example, a display, a touch screen, a mouse, a keyboard or button interface, a controller, and/or others, without limitation. For instance, FIG. 1 illustrates that the electroporation system 100 includes a user interface element implemented in the form of a graphical touch-screen user interface on a pulse generator 120. The user interface element is configured to display information related to operation of the electroporation system 100 and/or receive user input for facilitating control of the electroporation system 100 (e.g., to select parameters for, initiate, monitor, and/or end electroporation processes).

The electroporation system 100 includes various physical components that are usable to facilitate electroporation operations. For example, FIG. 1 illustrates that the electroporation system 100 includes a pulse generator 120 that is configured to supply electrical pulses to other components of the electroporation system 100. The pulse generator 120 may supply the electrical pulses via cable(s) 122, which may selectively connect the pulse generator 120 to one or more other components of the electroporation system 100. For example, the cable(s) 122 may connect the pulse generator 120 to a pipette docking assembly 121 to supply electrical current to target cells residing within a pipette tip of a pipette 130 connected to the pipette docking assembly 121. The pipette docking assembly 121 may include a pipette station 124 (to which the cable(s) 122 may connect), a pipette station guard 126, and a reservoir (e.g., a buffer tube 128, which receives the pipette 130). Additional aspects of components of the electroporation system 100 will be described in more detail hereinbelow.

Pipette Tips

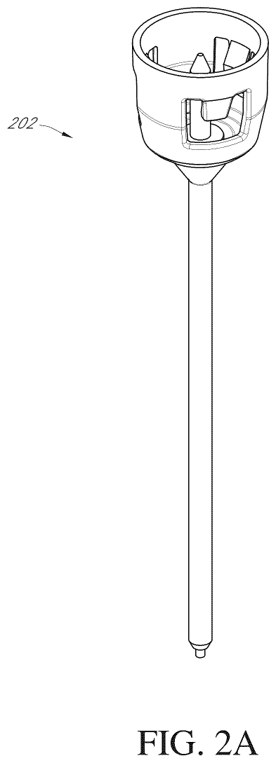

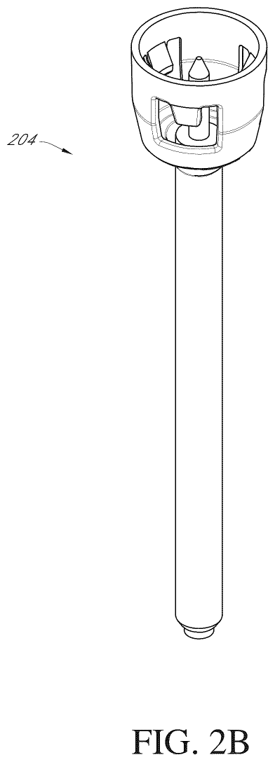

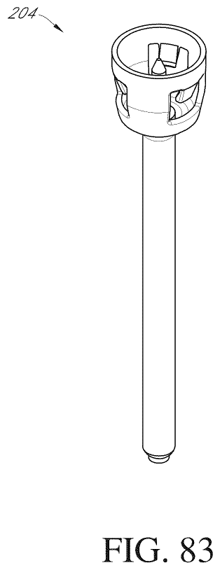

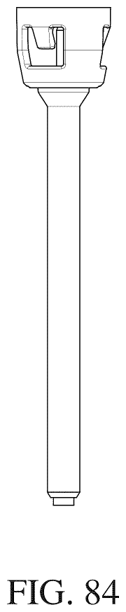

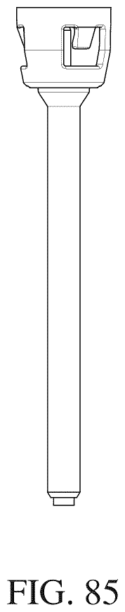

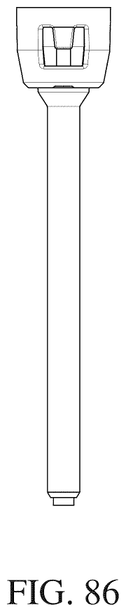

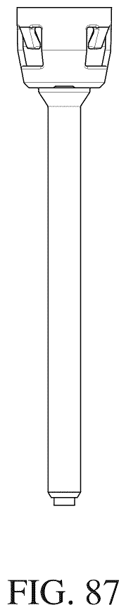

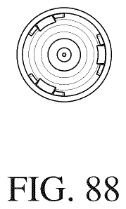

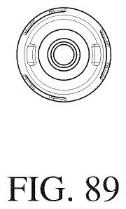

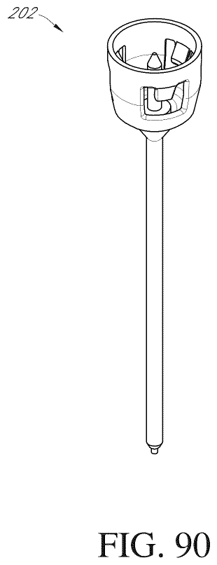

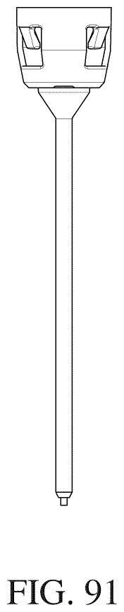

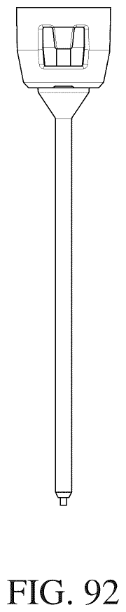

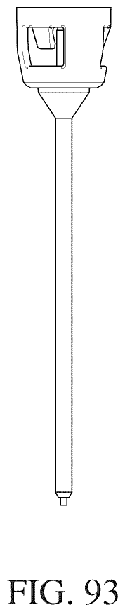

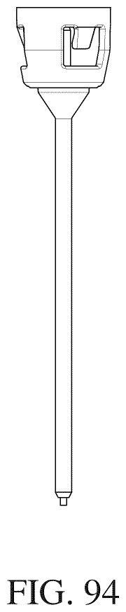

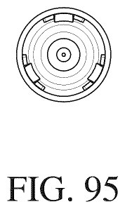

FIGS. 2A-7B and 83-96 illustrate aspects of pipette tips for use in the electroporation system of the disclosure. Such pipette tips may be connected to a pipette (e.g., pipette 130) and may hold a sample containing cells and a payload to facilitate electroporation. FIG. 2A depicts an example of a 10 μL pipette tip 202 (e.g., sized to hold a sample volume of about 10 μL) and FIG. 2B depicts a 100 μL pipette tip 204 (e.g., sized to hold a sample volume of about 100 μL). Although only 10 μL and 100 μL sizes are shown in FIGS. 2A and 2B, respectively, other sizes are within the scope of the present disclosure, including any pipette tips sized to contain a sample volume of between 0.1-500, 0.1-450, 0.1-400, 0.1-350, 0.1-300, 0.1-250, 0.1-200, 0.1-150, 0.1-100, 0.1-90, 0.1-80, 0.1-70, 0.1-60, 0.1-50, 0.1-40, 0.1-30, 0.1-20, 0.1-10, 0.0.1-5, 0.10-250, 0.10-100, 0.10-50, 1-500, 1-450, 1-400, 1-350, 1-300, 1-250, 1-200, 1-150, 1-100, 1-90, 1-80, 1-70, 1-60, 1-50, 1-40, 1-30, 1-20, 1-10, 1-10, 10-500, 10-450, 10-400, 10-350, 10-300, 10-250, 10-200, 10-150, 10-100, 10-90, 10-80, 10-70, 10-60, 10-50, 10-40, 10-30, 10-20 μL, including any increment in-between, including 0.2, 0.5, 1, 5, 10, 25, 50, 75, 100, 150, 200, 250, 300, 350, 400, 450 μL, and the like.

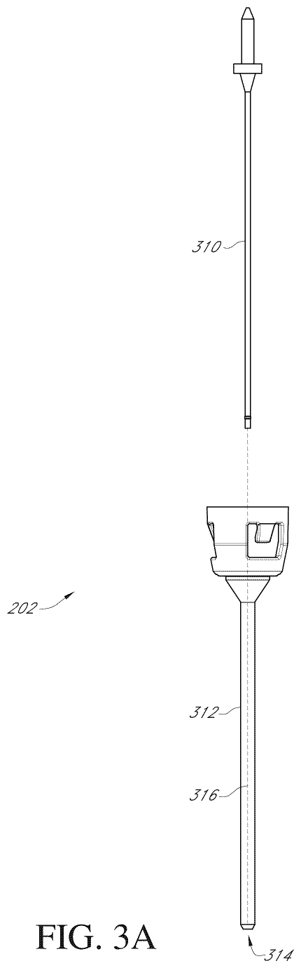

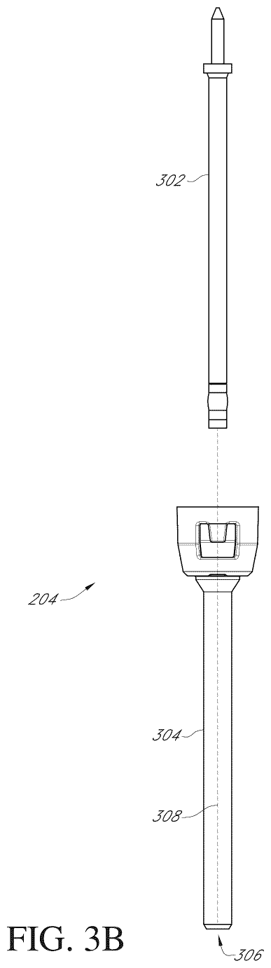

FIG. 3B shows a 100 μL pipette tip 204 may comprise a plunger 302 that is configured to be at least partially disposed within a lumen 308 of a tip sleeve 304 (FIG. 2B shows the plunger 302 fully inserted into the lumen 308 defined by the tip sleeve 304). The plunger 302 is configured to translate along the length of the lumen 308 to facilitate pipetting functions (e.g., aspirating and/or dispensing). For instance, the distal open end 306 of the lumen 308 may be positioned within a vessel containing a liquid sample including cells and a payload (e.g., nucleic acid, protein(s), etc.), and the plunger 302 may be drawn away (e.g., proximally) from the distal open end 306 of the lumen 308 to draw the cells and the payload into (e.g., aspirate) the lumen 308. The 100 μL pipette tip 204 may then be connected to other components of an electroporation system (e.g., the pipette docking assembly of FIG. 1) to electroporate the cells to introduce the payload into the cells.

At least a portion of the plunger 302 may comprise a conductive material to enable an electrical pulse to reach and/or travel through the contents of the lumen 308. For example, the plunger 302 may be coated with, formed from, or otherwise comprise a gold (e.g., gold plating), diamond-like carbon, conductive plastic, and/or any other conductive medical-grade materials (e.g., materials that are inert to mammalian cells).

FIG. 3A shows that the 10 μL pipette tip 202 may comprise a plunger 310 and tip sleeve 312 having a lumen 316 (with a distal open end 314), similar to the plunger 302 and lumen 308 of the 100 μL pipette tip 204.

For conventional pipette tips, a seal is created between the plunger and a lumen wall defining the lumen by a metal ring on the plunger that interfaces with the lumen wall. The amount of frictional force exhibited between the metal ring and the lumen wall can affect the push/pull force required to operate the pipette. The amount of frictional force exhibited between the metal ring and the lumen wall can be affected by the amount of interference between the metal ring and the lumen wall. By way of illustrative example, for a 10 μL tip, an interference within a range of 0 to 30 μm can give rise to a push/pull force within a range of 0 to 6 N to operate the pipette. For a larger tip, such as a 10 μL tip, an interference within a range of 0 to 10 μm can give rise to a push/pull force within a range of 0 to 6 N to operate the pipette. It can be difficult to consistently and reliably achieve an interference within the range of 0 to 10 μm in production, which can give rise to pipette tips (particularly larger pipette tips) that have an excessive interference between the metal ring and the lumen wall, leading to an excessive push/pull force necessary to operate the pipette (e.g., exceeding 6 N).

Accordingly, at least some pipette tips of the present disclosure may implement an alternative sealing component for creating a seal between the lumen wall and the plunger. In some embodiments, this is beneficial for pipette tips of larger sizes (e.g., 50 or 100 μL pipette tips, or larger).

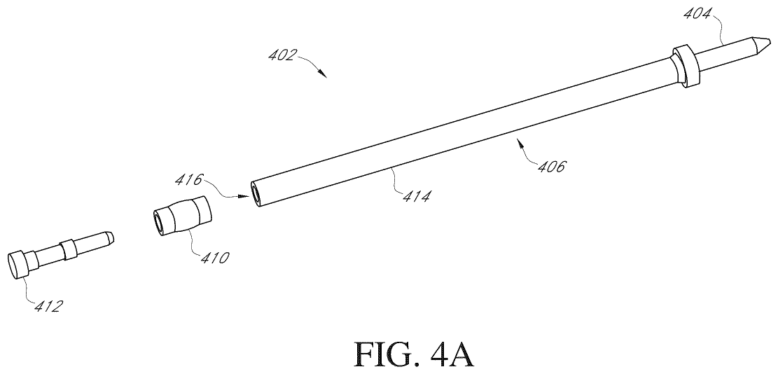

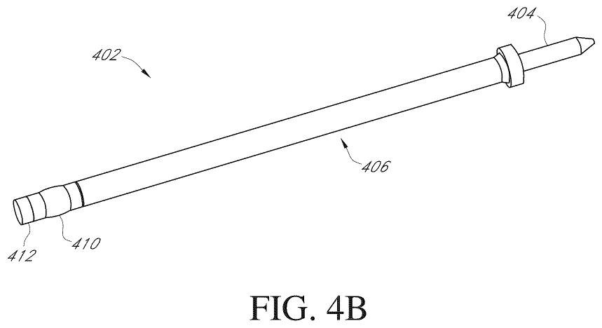

FIGS. 4A-4B illustrate an example plunger 402 of a 100 μL pipette tip (in both exploded (FIG. 4A) and assembled configurations (FIG. 4B)). In the embodiment of FIGS. 4A-4B, the plunger 402 includes an engagement section 404 and a lumen section 406. The engagement section 404 is configured to operably engage with a gripper jaw of a pipette configured to grasp and ungrasp the engagement section 404 to respectively retain and release the engagement section 404, as will be described in more detail hereinafter. The lumen section 406 is configured to be positioned within the lumen of the tip sleeve of a pipette tip and translate along the length of the lumen.

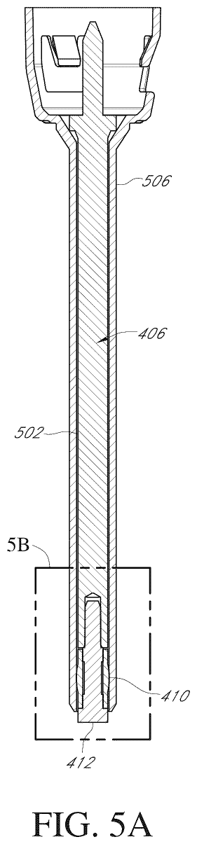

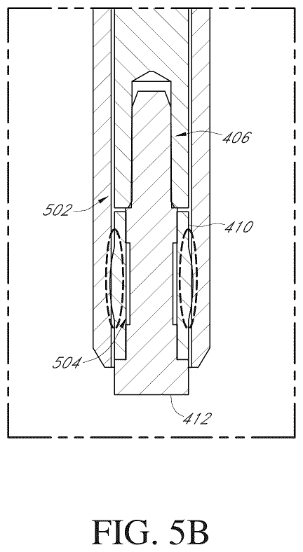

As shown in FIGS. 4A-4B, the lumen section includes a sealing component 410, which creates a seal between the plunger 402 and the lumen wall of the lumen within which the plunger is positioned. FIG. 5 shows a section view of the lumen section 406 of the plunger 402 positioned within a lumen of tip sleeve 506. FIG. 5B depicts an interference area between the sealing component 410 and the internal wall 502 of the tip sleeve 506 defining the lumen. Advantageously, the interference area does not extend along the entire length of the lumen section 406 of the plunger 402 that is positioned within the lumen, thereby facilitating reduced frictional force between the plunger 402 and the wall of lumen.

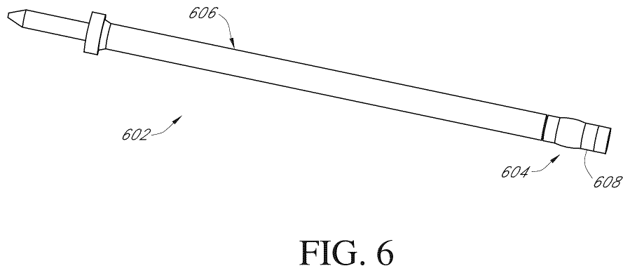

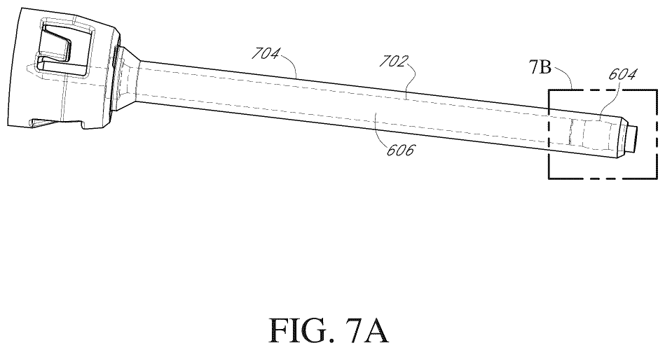

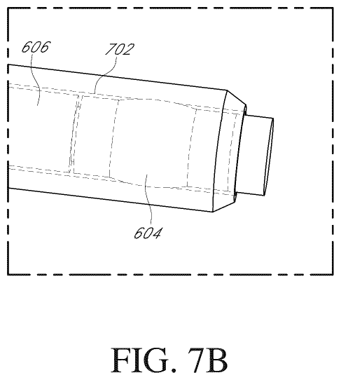

In the example of FIGS. 4A-4B and 5A-5B, the sealing component 410 is implemented as a polymer sleeve (other forms are possible, such as an O-ring design as shown in FIGS. 6 and 7A-7B). The sealing component 410 may comprise various types of materials, such as polytetrafluoroethylene (PTFE), other Teflon materials, and/or other pliable and biocompatible materials.

The sealing component 410 may be affixed to the lumen section 406 of the plunger 402 in various ways. In the example of FIGS. 4A-4B and 5A-5B, the lumen section includes a front pin 412 and a shaft section 414. The front pin 412 is configured to connect to the shaft section 414, such as by insertion of a portion of the front pin 412 into a retention hole 416 of the shaft section 414. The front pin 412 may further secure the sealing component 410 to the shaft section 414, such as by insertion of the front pin 412 through an opening in the sealing component 410 prior to entry of the front pin 412 into the retention hole 416 of the shaft section 414.

In some instances, a space is formed between at least a portion of the sealing component 410 and at least a portion of the lumen section 406 when the sealing component 410 is secured to the lumen section 406. This can contribute to the flexibility of the sealing component 410 for creating the seal between the lumen section 406 and the wall defining the lumen (e.g., reducing the frictional force therebetween while still maintaining the seal). FIG. 5B illustrates a space 504 formed between the sealing component 410 and the front pin 412 when the front pin 412 is inserted through the sealing component 410.

One will appreciate, in view of the present disclosure, that other methods for securing the sealing component 410 to the lumen section 406 may be implemented in accordance with the present disclosure (e.g., adhesive, mechanical fit, threaded connection, etc.).

As noted above, a sealing component of a plunger may take on various forms. FIGS. 6 and 7A-7B show an alternative form of a sealing component. FIG. 6 illustrates a plunger 602 where the sealing component is implemented as an O-ring 604, which may be coated (e.g., with an inert, lubricating material). The lumen section 606 of the plunger 602 includes a circumferential depression 608 configured to receive the O-ring 604. FIGS. 7A-7B shows the O-ring 604 interfacing with an internal wall 702 of a tip sleeve 704 defining a lumen to form a seal between the lumen section 606 and the internal wall 702. In some instances, an O-ring design may require more force than a polymer sleeve design to facilitate a seal between the plunger and pipette lumen (e.g., in view of the lack of an interior space in the O-ring design).

Pipettes and Pipette Assemblies

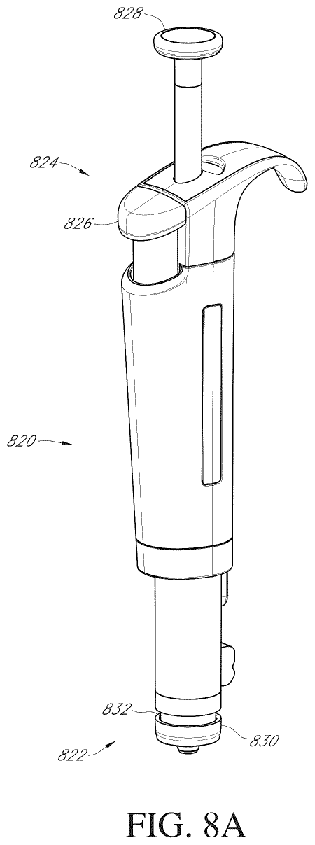

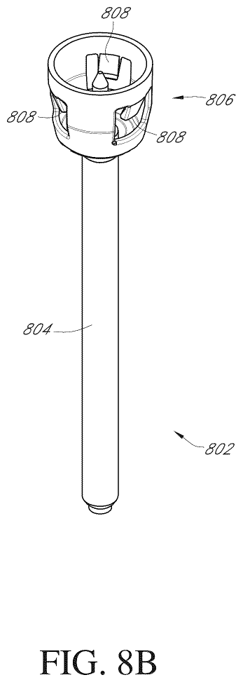

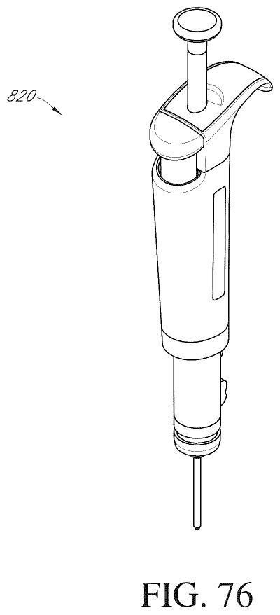

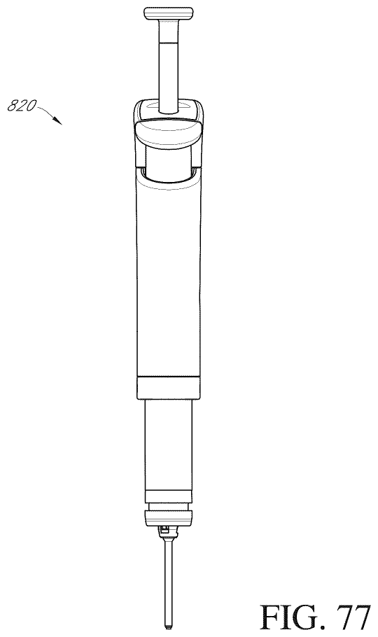

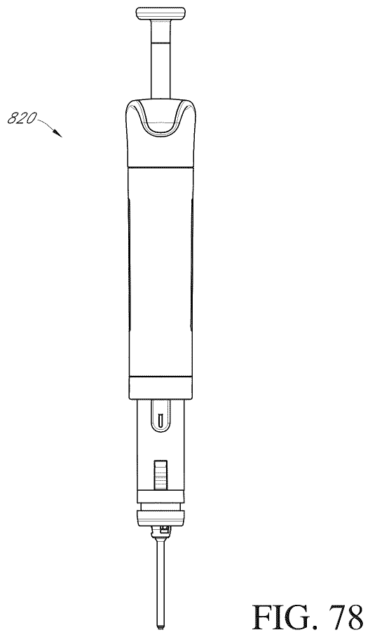

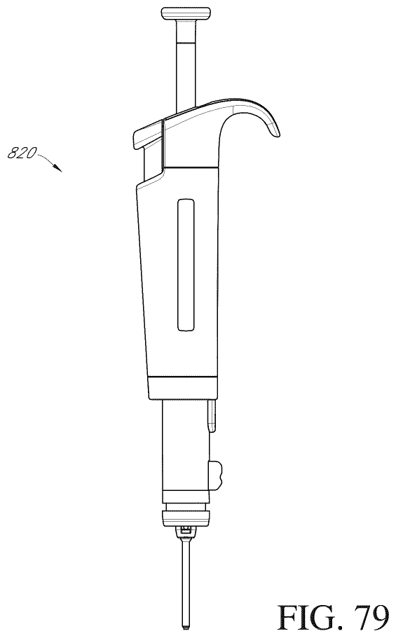

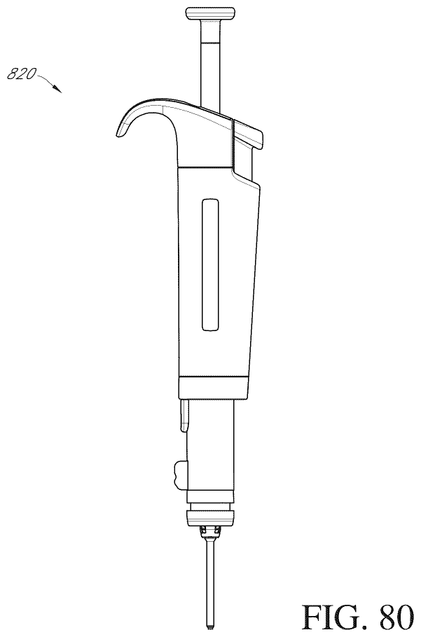

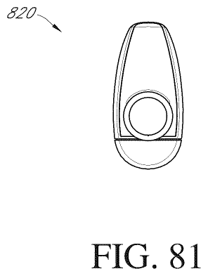

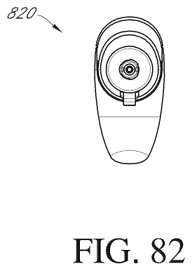

FIG. 8A illustrates a pipette 820 utilized in the system of the disclosure which is distally attachable to a pipette tip, such as the pipette tip illustrated in FIG. 8B. FIGS. 76-82 illustrate various views of the pipette 820 utilized in the system of the disclosure having a pipette tip attached. It will be appreciated that while the present disclosure illustrates an embodiment of a pipette configured to couple with a single pipette tip, pipettes of the disclosure may be configured to couple with 2, 3, 4, 5, 6, 7, 8, 9, 10, 11, 12, 13, 14, 15, 16 or more pipette tips while maintaining the same functionality (e.g., use of 2 proximally located actuators to control pipetting functions and tip attachment) by expanding the pipette design to include multiple channels having the same or similar structural features as disclosed herein for performing pipetting functions and attaching/detaching pipette tips including clip-tip connection, grasping/ungrasping plungers, and control movement of plungers to perform pipetting functions. As such, the pipette of the disclosure may be configured to reversibly couple multiple pipette tips, the functionality of which is controlled by 2 actuators proximally disposed on the pipette.

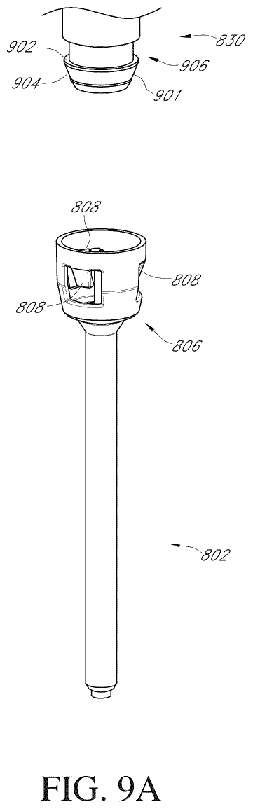

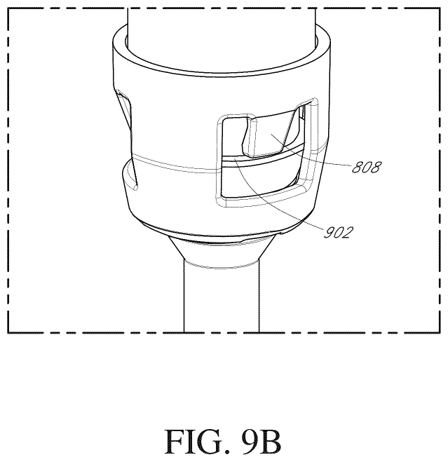

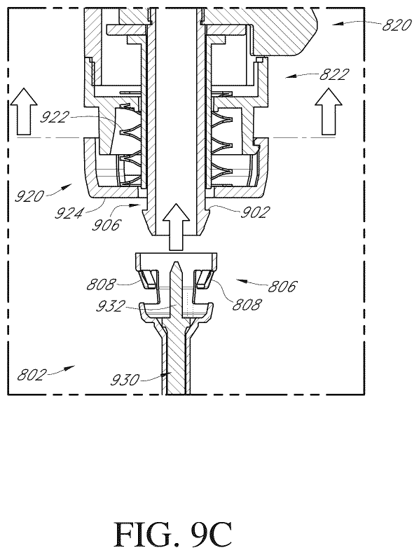

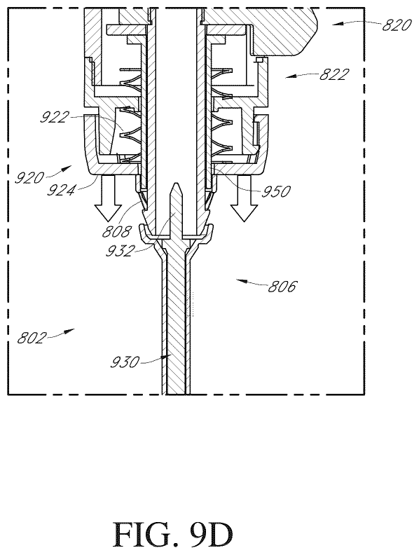

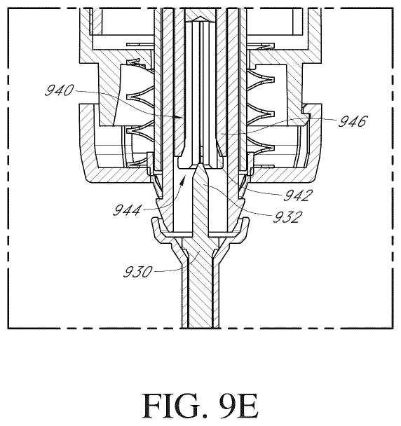

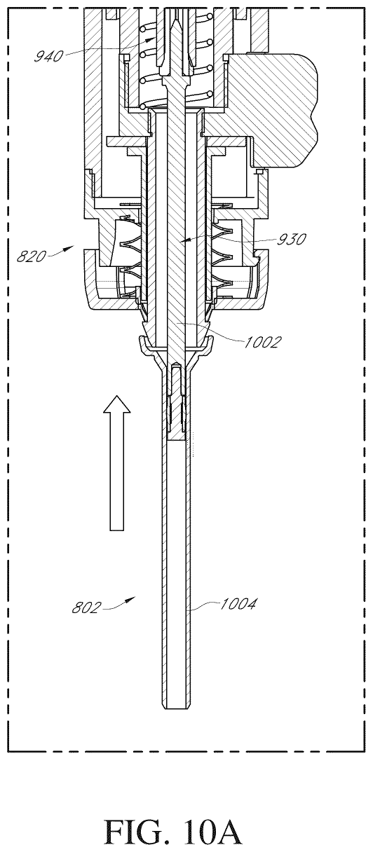

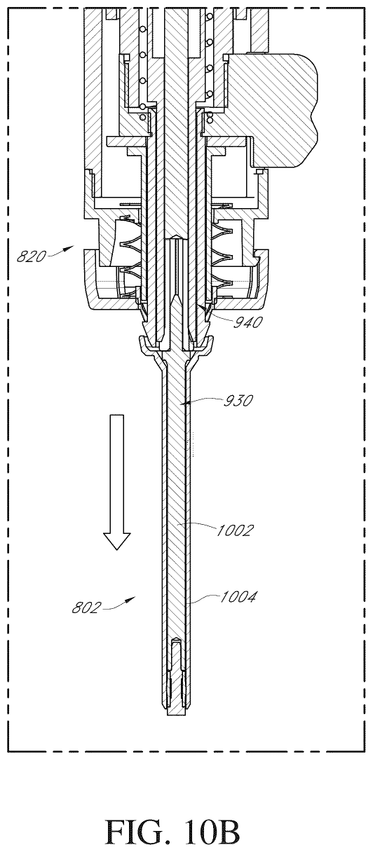

In various embodiments, the pipette includes a proximal section having a handle, and a distal section configured to reversibly attach to a pipette tip. The pipette further includes a first actuator and a second actuator which are operable to control functionality of the pipette. In some embodiments, the first actuator is disposed in the proximal section, and when actuated, is operable to control: i) a pipetting function (aspiration and dispensing of a sample) of the pipette; and ii) grasping and ungrasping of a plunger disposed within a lumen of the pipette tip (see, for example, FIGS. 9A-10B). In related embodiments, the second actuator is disposed in the proximal section, and when actuated, is operable to cause the tip sleeve of the pipette tip to detach from the distal section of the pipette. In various embodiments, both the first and second actuators are actuated by depressing and/or unpressing the actuators. It will be appreciated that each of the first and second actuators are oriented such that operation/control of the respective functions of each are controllable by the thumb of a user grasping the handle of the pipette. The pipette also includes a pipette electrode which contacts an electrode disposed on the docking station when the pipette is docked in the pipette docking assembly (see, e.g., FIG. 1).

As discussed and illustrated further herein, in embodiments, the first actuator has a first undepressed position and a second partially depressed position in which the actuator is moved distally with respect to the handle. When a pipette tip is attached and the plunger grasped by the gripper jaw, transitioning the first actuator from the first undepressed position to the second partially depressed position causes dispensing from the pipette tip by translation of the plunger within the lumen of the pipette tip and transitioning of the first actuator from the second partially depressed position to the first undepressed position causes aspirating into the pipette tip by translation of the plunger within the lumen of the pipette tip.

As discussed further herein, the first actuator has a third fully depressed position in which the first actuator is advanced distally with respect to the handle past the second partially depressed position. When a plunger is grasped by the gripper jaw, transitioning the first actuator from the second partially depressed position to the third fully depressed position causes opening of the gripper jaw and ungrasping of the plunger. Transitioning the first actuator from the third fully depressed position to the second partially depressed position causes grasping of the plunger. During attachment of a pipette tip to the pipette, the first actuator is transitioned to the third fully depressed position, the engagement section of a plunger is oriented into a distal opening of the gripper jaw, and the first actuator is then transitioned to the second partially depressed position to effectuate closing of the gripper jaw and grasping of the engagement section such that aspirating and dispensing functions are controlled by transition of the first actuator between the first and second positions via translation of the plunger with the lumen of the pipette tip. During detachment of a pipette tip to the pipette, the first actuator is transitioned to the third fully depressed position to ungrasp and release the engagement section of the plunger and may be maintained in the third position until detachment of the tip sleeve is effectuated if it has not already been detached by actuation of the second actuator as discussed further herein.

As discussed further herein with reference to particular Figures, in various embodiments, the pipette includes a gripper mechanism operably coupled to the first actuator which has a gripper jaw and a gripping sleeve disposed about the gripper jaw. In some embodiments, operation of the gripper mechanism is controlled by first actuator and transitions between a closed configuration (to grasp) and an open configuration (to ungrasp) upon transitioning of the first actuator between the second partially depressed position and the third fully depressed position. The gripper jaw includes a jaw opening for receiving an engagement section of the plunger and is operable to grasp the engagement section of the plunger when the first actuator is in the first undepressed position and the second partially depressed position via relative positioning with the gripper sleeve. For example, the gripping sleeve is positioned around the gripper jaw and configured to exert an inward force on the gripper jaw to cause the gripper jaw to exert a compressive force on the engagement section of the plunger to retain the engagement section of the plunger within the jaw opening when the first actuator is in the first depressed position or the second partially depressed position.

In embodiments, actuation of the second actuator causes detachment of the tip sleeve of the pipette tip, separate from operation of the first actuator to control plunger engagement and movement. In some embodiments, the second actuator has a first undepressed position and a second depressed position in which the actuator is moved distally toward the distal section of the pipette. In further embodiments, a tip ejection sleeve is operably connected to the second actuator and disposed adjacent to the tip interface of the pipette such that the tip ejection sleeve is moved distally with respect to the tip interface when the second actuator is actuated by transitioning the second actuator from the first undepressed position to the second depressed position causing displacement and detachment of the tip sleeve from the distal section of the pipette.

As discussed throughout, a pipette tip may be selectively attached to a pipette of the disclosure. FIG. 8B illustrates a pipette tip 802, which may correspond to any pipette tip discussed herein. FIG. 8A shows a pipette 820. In the example of FIGS. 8A-8B, the pipette tip 802 includes an attachment interface 806 adjacent to the lumen thereof 804. The attachment interface 806 includes tabs 808, which are configured to engage with corresponding attachment features of the pipette 820 (any number of tabs may be utilized). In embodiments, the tabs are angled toward the lumen of the pipette tip 802. The corresponding attachment features of the pipette 820 are arranged on a distal section 822 of the pipette 820 as a tip interface 830 including a retention platform 832. The distal section 822 of the pipette may comprise a section of the pipette 820 that is opposite to a proximal section 824 of the pipette 820 that includes actuators for manual control to operate the pipette 820 (e.g., first actuator 828 and second actuator 826, to control pipetting functionality and plunger engagement, and tip sleeve detachment/ejection, respectively).

FIG. 9A shows example attachment features of the pipette 820, e.g., the tip interface, and corresponding attachment features of the pipette tip 802, e.g., the attachment interface. FIG. 9A, shows the corresponding attachment features separated, e.g., the pipette tip and pipette detached from one another. FIG. 9B shows the corresponding attachment features coupled, e.g., the pipette tip sleeve attached to the pipette. In particular, FIG. 9A illustrates a retention platform 902 of the tip interface 830 of the distal section 822 of the pipette. The retention platform 902 resides atop a curved surface 904 and within a recess 906 of the distal section 822. This allows the tabs 808 of the pipette tip 802 to advance into engagement with the retention platform 902. For example, when the distal section 822 of the pipette 820 is pressed into the attachment interface 806 of the pipette tip 802, the tabs 808 of the attachment interface may advance and expand over the angled surface 904 until reaching the recess 906, at which time the tabs 808 may retract inward toward the recess 906 and into engagement with the retention platform 902.

In some instances, after the tabs 808 reach the recess 906 and are retracted thereinto, a biasing member of the pipette 820 may operate to bias the tabs 808 into engagement with the retention platform 902. FIG. 9C shows a biasing member 920 of the distal section 822 of the pipette 820, which includes a spring 922 and a biasing platform 924. FIG. 9C shows the attachment interface 806 of the pipette tip 802 arranged ready for connection to the distal section 822 of the pipette 820. As the attachment interface 806 advances over the distal section 822 of the pipette 820 (per the arrows shown in FIG. 9C), the attachment interface 806 presses and moves the biasing platform 924 to allow the tabs 808 to reach the retention platform 902. The spring 922 responsively becomes compressed such that after pressing of the attachment interface 806 into the distal section 822 of the pipette 820 ceases (after the tabs 808 have reached the recess 906), the spring 922 forces the biasing platform 924 against the attachment interface 806 to force the tabs 808 thereof into engagement with the retention platform (as shown in FIG. 9D and indicated by the bold arrows thereof).

FIG. 9D illustrates the plunger 930 of the pipette tip 802. The plunger 930 (in particular an engagement section 932 of the plunger 930) may become reversibly gripped by a gripper jaw, the proximal region of which is surrounded by a gripping sleeve, the gripper jaw and gripping sleeve forming a gripper mechanism being disposed in the distal section 822 of the pipette 820. FIG. 9E shows the gripper mechanism 940 including the gripper jaw 942 and the gripping sleeve 946. The gripper jaw 942 includes a distally disposed jaw opening 944 for receiving the engagement section 932 of the plunger 930 such that it can be retained (e.g., grasped) by the gripper jaw 942. In some implementations, the gripper jaw 942 comprises an at least partially flexible material to enable insertion of the engagement section 932 thereinto. Sloping or curvature at the top portion of the engagement section 932 of the plunger 930 may improve ease of insertion of the engagement section into the gripper jaw 942.

The gripper mechanism 940 is operably connected to the first actuator 828 and actuatable by the first actuator to facilitate advancement of the gripper jaw 942 into engagement with the engagement section 932 of the plunger 930. For example, the gripper mechanism may be actuated by operation of the first actuator 828 (see FIG. 8A) of the pipette 820. In embodiments, the gripper sleeve 946 in unison with the gripper jaw 942 may be advanced distally for a distance during actuation of the first actuator (e.g., from the first undepressed position to the second partially depressed position). Subsequently, only the gripper jaw 942 is advanced during further actuation to open the gripper jaw 942 to receive the engagement section 932 (e.g., from the second partially depressed position to the third fully depressed position) which is coordinated with simultaneous advancement of the tabs 808 of the attachment interface 806 of the pipette tip 802 into engagement with the retention platform 902 (e.g., by holding down the first actuator 828 while pressing the pipette 820 and the pipette tip 802 into one another). Alternatively, the gripper sleeve 946 in unison with the gripper jaw 942 may be advanced distally for a distance during actuation of the first actuator (e.g., from the first undepressed position to the second partially depressed position), and subsequently, only the gripper jaw 942 is advanced during further actuation to open the gripper jaw 942 to receive the engagement section 932 (e.g., from the second partially depressed position to the third fully depressed position), and asynchronously with actuation of the first actuator and engagement of the gripper jaw 942 with the engagement section 932, the tabs are advanced into engagement with the retention platform 902 (e.g., by first pressing the tabs 808 into engagement with the retention platform 902 and subsequently depressing the first actuator 828 and translating the first actuator from the first undepressed position to the third fully depressed position).

The plunger mechanism 940 also includes a gripping sleeve 946 positioned around the gripper jaw 942 as illustrated, for example, in FIG. 9E. The gripping sleeve 946 is configured to exert an inward force on the gripper jaw 942 to cause the gripper jaw 942 to exert an inward compressive force on the engagement section 932 to grasp and retain the engagement section 932 of the plunger 930 within the gripper jaw 942. In some implementations, the gripping sleeve 946 comprises a material that is at least partially more rigid than material of the gripper jaw 942.

After gripping the plunger 930, the gripper mechanism 940 may be actuated by operation of the first actuator 828 (see FIG. 8A) of the pipette 820 to facilitate pipetting functionality. FIGS. 10A and 10B show the gripper mechanism 940 being translated in an aspirating direction toward the proximal section of the pipette (see FIG. 10A, as indicated by the arrow therein, e.g., resulting from movement of the first actuator from the second partially depressed position toward the first undepressed position) and in a dispensing direction toward the distal section of the pipette (see FIG. 10B, as indicated by the arrow therein, e.g., resulting from movement of the first actuator from the second partially depressed position toward the first undepressed position). With the plunger 930 gripped by the gripper jaw 942 of the gripper mechanism 940, actuation of the gripper mechanism 940 causes translation of the lumen section 1002 of the plunger 930 within the lumen 1004 of the pipette tip 802.

In some embodiments, after the pipette tip 802 is engaged with the pipette 820 (e.g., with the tabs 808 engaged with the retention platform 902 and with the plunger 930 engaged with the gripper mechanism 940), the pipette tip 802 may be selectively ejected from the pipette 820 via a two-step process, which may reduce the peak amount of force needed to facilitate the disengagement (as compared to a single-action process for facilitating disengagement). By way of non-limiting example, whereas a single-action ejection process may require a peak force of about 60 N, a multi-step ejection process as presently disclosed may require a peak force of about 40 N. As noted above, the pipette 820 includes a second actuator 826 (see FIG. 8A) which may be used to cause the attachment interface 806 to disengage from the distal section 822 of the of the pipette 820 (without causing the gripper mechanism 940 to disengage from the plunger 930).

For example, the second actuator 826, when pressed, may cause actuation of a tip ejection sleeve 950 (see FIG. 9D) to cause the tip ejection sleeve 950 to advance toward the tabs 808 (e.g., downward) from an inner side of the tabs 808. During advancement, the tip ejection sleeve 950 may press on the inner side of the tabs 808 to bend the tabs 808 outward and cause the tabs 808 to disengage from the retention platform 902. The biasing member 920 may then press the attachment interface 806 of the pipette tip 802 downward from the pipette 820, thereby ejecting the attachment interface 806 (and the lumen attached thereto) of the pipette tip 802 from the pipette.

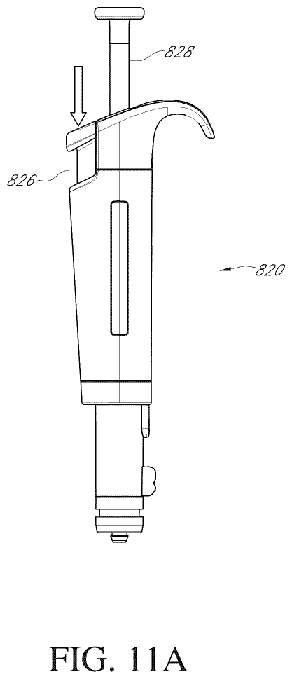

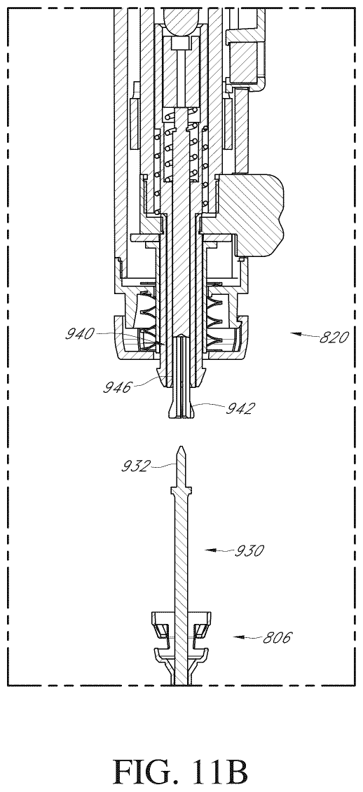

FIG. 11A is a schematic depiction of actuating (e.g., depressing) the second actuator 826 of the pipette 820 to cause ejection of the attachment interface (e.g., attachment interface 806) and lumen (e.g., lumen 1004) of the pipette tip (e.g., pipette tip 802) from the pipette 820. In the example of FIG. 11A, the second actuator 826 is configured to traverse a blank travel distance when depressed to move the second actuator from a first undepressed position to a second depressed position (e.g., a distance of up to about 6 mm) prior to causing disengagement of the attachment interface 806 and lumen of the pipette tip from the pipette 820 by the tip ejection sleeve (which occurs by pressing the ejection button 826 through a final distance of about 2.5 mm by applying about 20 to 30 N of force). Such functionality may prevent inadvertent ejection of the attachment interface and lumen of the pipette tip from the pipette 820 (other blank travel and ejection distances may be used).

FIG. 11B is a schematic depiction of a pipette with the attachment interface and lumen of the pipette tip ejected therefrom. In embodiments, the first actuator is actuated to release the plunger from the gripper mechanism before actuation of the second actuator to disengage the attachment interface. In some embodiments, the second actuator is first actuated to detach the attachment interface, and subsequently the first actuator is actuated to release the plunger from the gripper mechanism. As depicted in FIG. 11B, depressing the first actuator 828 after disconnection of the attachment interface 806 from the pipette 820 causes ejection of the engagement section of the plunger 930 from the gripper mechanism 940 of the pipette. For instance, depressing of the first actuator 828 after disconnection of the attachment interface 806 from the pipette 820 may cause the gripper jaw 942 to advance out of the gripping sleeve 946, thereby releasing the inward forces previously exerted by the gripper jaw 942 on the engagement section 932 of the plunger 930 and ejecting the plunger 930 from the gripper mechanism 940. In the embodiment shown in FIG. 11B, example forces associated with a plunger spring of about 12.32 N and a gripper spring of about 28.20 N, can be overcome by depressing the first actuator 828 to facilitate plunger ejection (e.g., resulting in a total force of about 40.52 N experienced by the user).

In various aspects, the disclosure provides pipette assemblies including a pipette reversibly attached to a pipette tip. In some embodiments, the pipette of the assembly includes a proximal section having a handle, a distal section having a tip interface, and a first actuator disposed in the proximal section. The pipette tip reversibly attached to the pipette of the assembly includes a tip sleeve defining a lumen extending from a proximal end of the pipette tip to a distal end of the pipette tip, a plunger at least partially disposed within the lumen, and an attachment interface disposed at the proximal end of the pipette tip. In various embodiments, the plunger is reversibly operably coupled to the first actuator, and when operably coupled, performs a pipetting function upon actuation of the first actuator by translating along the lumen. Additionally, the tip sleeve is reversibly attached to the distal section via one or more tabs of the attachment interface engaged with a retention platform of the distal section. In embodiments, the pipette of the assembly further includes a pipette electrode disposed in the distal section and electrically coupled to the plunger when the plunger is operably coupled to the first actuator.

In some embodiments, the pipette of the assembly includes a proximal section having a handle, a distal section having a tip interface, a first actuator disposed in the proximal section, and a gripper mechanism disposed in the distal section having a gripper jaw. The pipette tip reversibly attached to the pipette of the assembly includes a tip sleeve defining a lumen extending from a proximal end of the pipette tip to a distal end of the pipette tip, and a plunger at least partially disposed within the lumen. In various embodiments, the plunger is reversibly operably coupled to the first actuator via the gripper jaw, and when operably coupled, performs a pipetting function upon actuation of the first actuator by translating along the lumen. In embodiments, the pipette of the assembly further includes a pipette electrode disposed in the distal section and electrically coupled to the plunger when the plunger is operably coupled to (e.g., grasped by) the gripper jaw.

Pipette Docking Assemblies

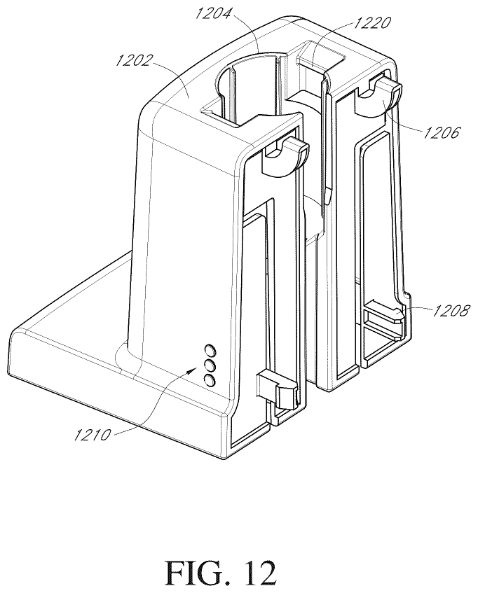

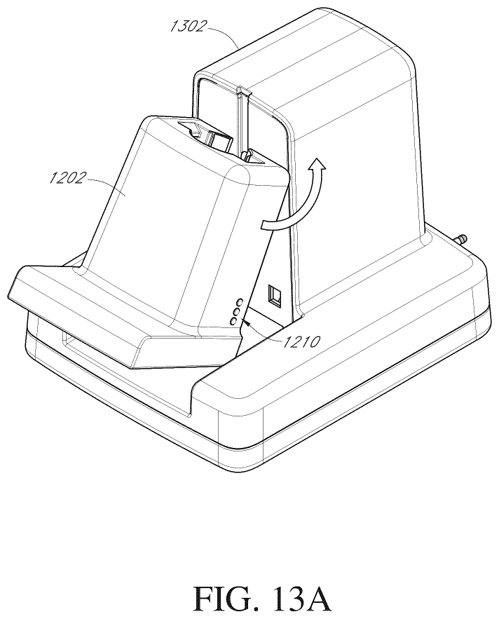

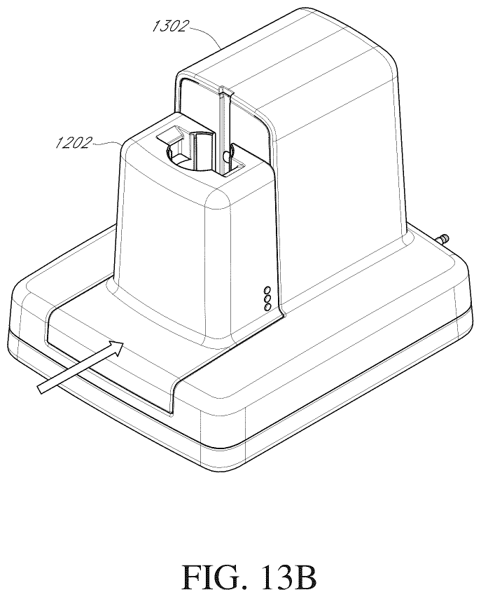

FIGS. 12 and 13 illustrate aspects of an example pipette station guard of an electroporation system (e.g., electroporation system 100).

FIG. 12 illustrates a pipette station guard 1202. The pipette station guard 1202 may be affixed to a pipette station to protect users against potential electrical shock. The pipette station guard 1202 may additionally include a reservoir opening 1204 (e.g., buffer tube opening), for receiving a reservoir (e.g., buffer tube), which can receive a pipette (e.g., pipette 820) and/or components connected thereto (e.g., a pipette tip).

To facilitate connection to a pipette station, the pipette station guard 1202 may comprise various connection elements, such as one or more locking hooks that are configured to engage with one or more corresponding hook catches of the pipette station. Such locking hook(s) may take on various forms. For instance, the example of FIG. 12 illustrates the pipette station guard 1202 as including one or more pivot hooks 1206, which are configured to rotate into engagement with one or more hook catches (e.g., pivot hook catches) of the pipette station (see FIG. 13). The example of FIG. 12 also illustrates the pipette station guard 1202 as including one or more flexible hooks 1208 configured to advance into engagement with one or more hook catches of the pipette station (see FIG. 13).

FIG. 12 also illustrates the pipette station guard 1202 further comprises one or more finger guides 1210 that indicate one or more pressable surfaces of the pipette station guard 1202. The pressable surface(s) of the pipette station guard 1202 are pressable for causing movement of the one or more flexible hooks 1208 to facilitate engagement/disengagement of the one or more flexible hooks 1208 with/from their corresponding hook catch(es) of the pipette station (see FIG. 13).

In the example of FIG. 12, the pipette station guard 1202 includes a pair of pivot hooks 1206 and a pair of flexible hooks 1208 arranged on the rear surface of the pipette station guard 1202. The pair of pivot hooks 1206 is arranged on the top portion of the rear surface, and the pair of pivot hooks is arranged on the bottom portion of the rear surface. FIG. 12 also depicts the finger guides 1210 as arranged on bottom portions of lateral surfaces of the pipette station guard 1202 (an opposing finger guide is arranged on the bottom portion of the opposing lateral surface of the pipette station guard 1202).

FIG. 13 illustrates the pipette station guard 1202 being moved into engagement with a pipette station 1302. FIG. 13A shows the pivot hooks 1206 of the station guard 1202 being initially inserted into corresponding pivot hook catches 1304 of the pipette station 1302. After insertion of the pivot hooks 1206, the station guard 1202 is rotated (indicated by the rotational arrow in FIG. 13A) toward the pipette station 1302 until the flexible hooks 1208 reach and interlock with corresponding flexible hook catches 1306. FIG. 13A also illustrates the finger guides 1210 of the pipette station guard 1202, which may be pressed to move the flexible hooks 1208 inward toward one another to enable the flexible hooks 1208 to easily enter openings associated with the flexible hook catches 1306. After entry of the flexible hooks 1208 into the openings, pressure may be released from the finger guides 1210 to allow outward movement of the flexible hooks 1208 to cause the flexible hooks 1208 to interlock with the flexible hook catches 1306. FIG. 13B shows the pipette station guard 1202 being fully mounted to the pipette station 1302.

In some embodiments, to remove the pipette station guard 1202 from the pipette station 1302, a user may press on the finger guides 1210 to allow the flexible hooks to disengage from the flexible hook catches 1306 and withdraw/rotate the pipette station guard 1202 out of engagement with the pipette station 1302.

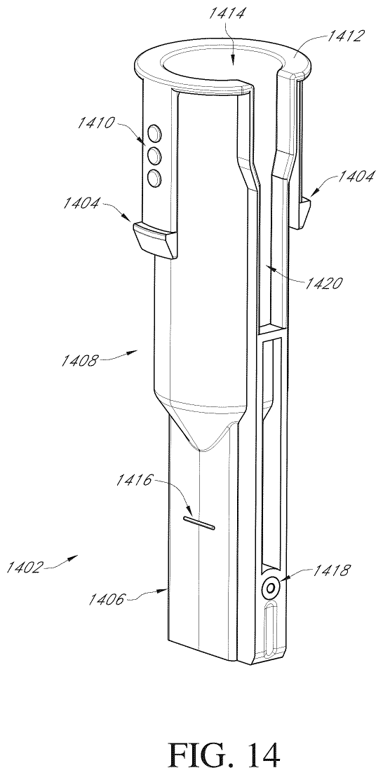

As noted above, the pipette station guard 1202 may comprise a reservoir opening 1204 for receiving a reservoir. FIG. 14 illustrates an example reservoir 1402 (e.g., buffer tube), which may be inserted into a reservoir opening 1204 of a pipette station guard 1202 (e.g., when the pipette station guard 1202 is mounted to a pipette station 1302). The reservoir 1402 of FIG. 14 includes latching members 1404, which are configured to engage with corresponding latch catches 1220 of the pipette station guard 1202 (see FIG. 12) when the reservoir 1402 is inserted into the reservoir opening 1204 of the pipette station guard 1202. The latching members 1404 of the reservoir 1402 can enable the reservoir 1402 to connect to the pipette station guard 1202 in a manner that prevents the reservoir 1402 from becoming inadvertently disconnected from the pipette station guard 1202 during use (e.g., while withdrawing pipette components from the reservoir 1402, thereby mitigating potential spillage and/or damage to pipette components).

In some instances, the shape of the reservoir 1402 itself can contribute to mitigating the risk of damage to pipette tips. For example, the reservoir 1402 may comprise a lower section 1406 in which a pipette tip is configured to reside and an upper section 1408 into which a distal section of a pipette is configured to reside. The shape of the upper section 1408 may enforce entry and withdrawal of the pipette in a manner that is longitudinally aligned with the reservoir 1402, thereby enforcing alignment of the pipette tip with the lower section 1406 and preventing damage thereto during insertion and/or withdrawal.

In the example of FIG. 14, the latching members 1404 are arranged on an exterior of the reservoir 1402. FIG. 14 also shows that the latching members 1404 may include respective finger cues 1410 for guiding positioning of user fingers for applying force to the one or more latching members 1404. Application of force on the latching members 1404 from the positioning of the finger cues 1410 may cause flexing/deformation of the latching members 1404, thereby enabling them to engage with or disengage from the corresponding latch catches 1220 of the pipette station guard 1202. The finger cues 1410 can also guide user finger placement when handling the reservoir so as to mitigate the risk of human fingers contacting pipette components during insertion and/or withdrawal of pipette components.

In the example of FIG. 14, the latching members 1404 extend from a flange 1412 of the reservoir 1402, which extends around a pipette opening 1414 of the buffer tube 1402 (for receiving pipette components). The flange 1412 may be at least partially sloped inward toward the pipette opening 1414 to direct any fluids that fall from pipetting components into the pipette opening. The flange 1412 may also operate to provide space between the finger cues 1410 and the pipette opening 1414, thereby contributing to mitigation of the risk of contamination of pipette components (e.g., during insertion and/or withdrawal of pipette components through the pipette opening 1414).

FIG. 14 also shows that the reservoir 1402 may comprise a volume indicator 1416 to allow users to ascertain when the reservoir 1402 contains a sufficient quantity of buffer solution to perform electroporation. The reservoir 1402 of FIG. 14 also includes an electrode 1418, which may contact a corresponding electrode of a pipette station (or pipette station guard) to allow electrical pulses from a pulse generator to reach the buffer solution within the reservoir 1402 (and target cells within a pipette tip positioned within the buffer solution). In some instances, the reservoir 1402 includes one or more hooks or other retention members for retaining pipette components therein (e.g., a pipette and/or pipette tip).

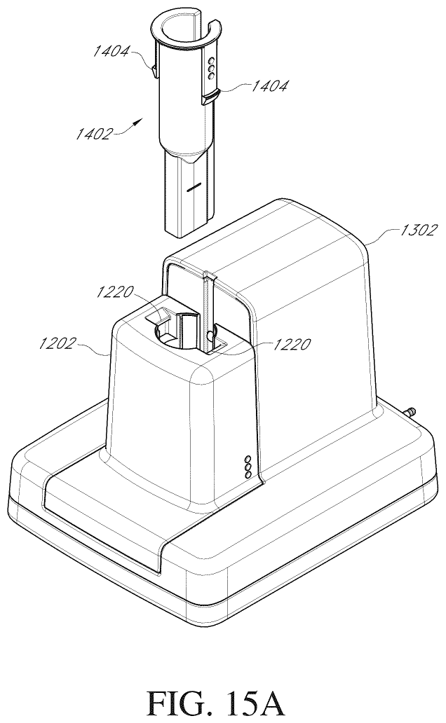

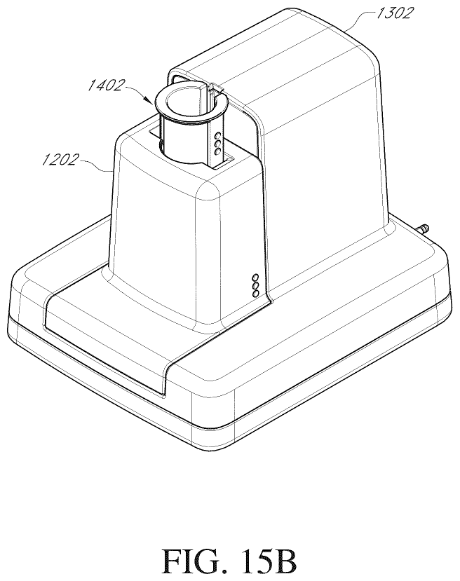

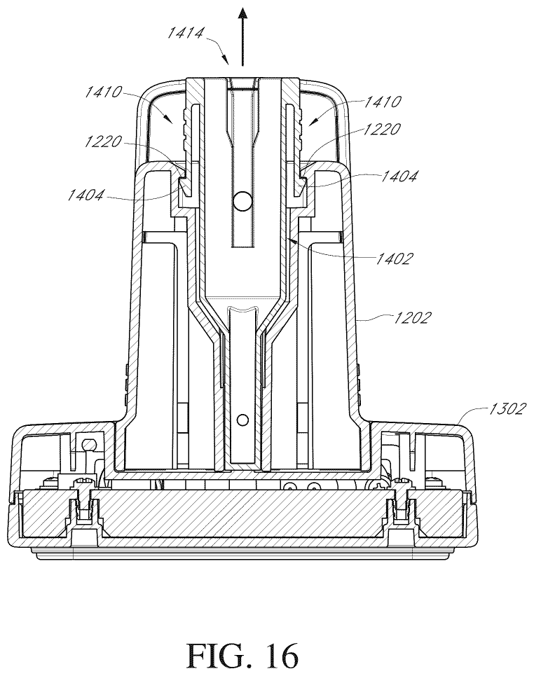

FIGS. 15A and 15B illustrate the reservoir 1402 being inserted into the pipette station guard 1202 that is mounted to the pipette station 1302. As noted previously, the latching members 1404 of the reservoir 1402 interlock with corresponding latch catches 1220 of the pipette station guard 1202. FIG. 16 illustrates a sectional view of the reservoir 1402 positioned within the reservoir opening 1204 of the pipette station guard 1202, with the latching members 1404 of the reservoir 1402 interlocked with the corresponding latch catches 1220 of the pipette station guard 1202. As is evident from FIG. 16, the force application directions for causing the latching members 1404 to disengage from the corresponding latch catches 1220 (inward from the finger cues 1410) are different from the pipette withdrawal direction (indicated by the bolded arrow in FIG. 16) for withdrawing pipette components from the pipette opening 1414 of the reservoir 1402. As noted above, this may mitigate the risk of the reservoir 1402 inadvertently withdrawing from the reservoir opening 1204 of the pipette station guard 1202 when withdrawing pipette components from the reservoir 1402.

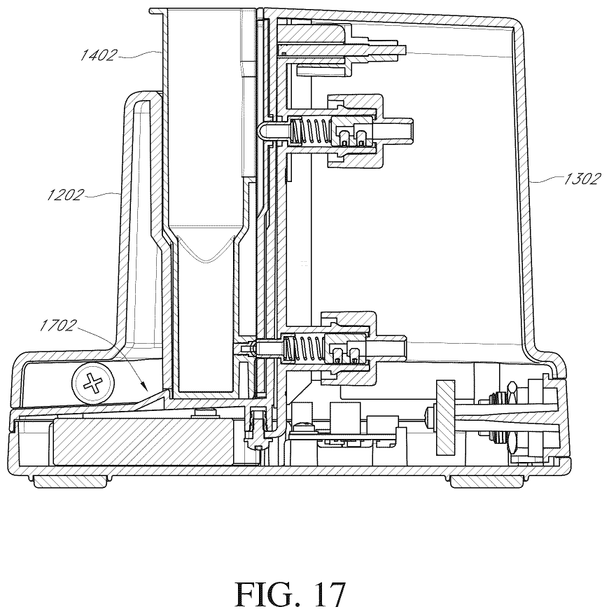

FIG. 17 illustrates a safety interlock feature 1702 of the pipette station 1302, which is configured to engage/interact with a portion of the reservoir 1402 (e.g., a bottom portion thereof) when (i) the pipette station guard 1202 is connected to the pipette station 1302 and (ii) the latching members 1404 of the reservoir 1402 are engaged with the corresponding latch catches 1220 of the pipette station guard 1202. In the example of FIG. 17, the safety interlock feature 1702 is implemented as an upward protrusion that prevents the reservoir 1402 from passing through the protrusion (e.g., in the direction used for withdrawal of the pipette station guard 1202). Thus, with the reservoir 1402 connected to the pipette station guard 1202, the safety interlock feature 1702 may prevent inadvertent removal of the pipette station guard 1202 from the pipette station 1302.

The height of the reservoir 1402 (which interfaces with a vertical wall of the pipette station 1302 as shown in FIG. 17) may further contribute to preventing inadvertent removal of the pipette station guard 1202 from the pipette station 1302 (e.g., by preventing the rotation necessary to remove the pipette station guard 1202 from the pipette station 1302).

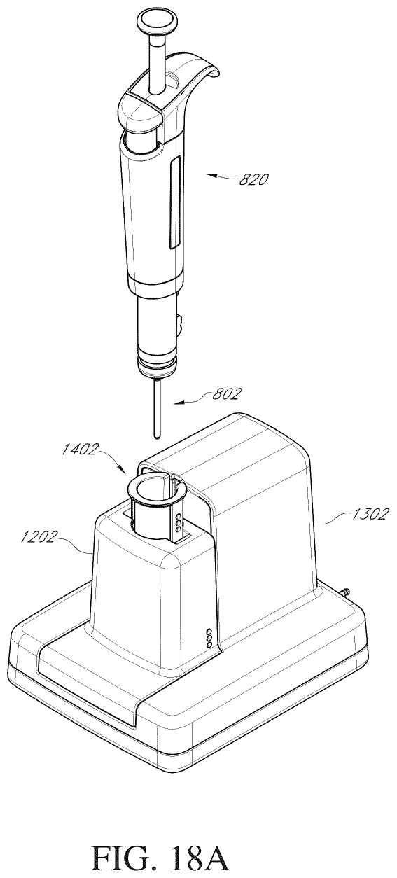

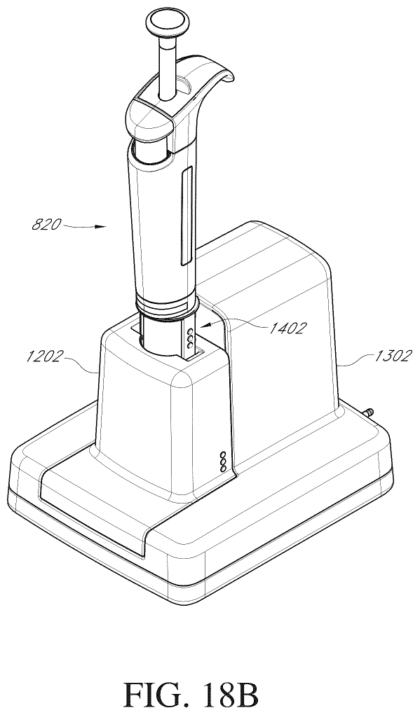

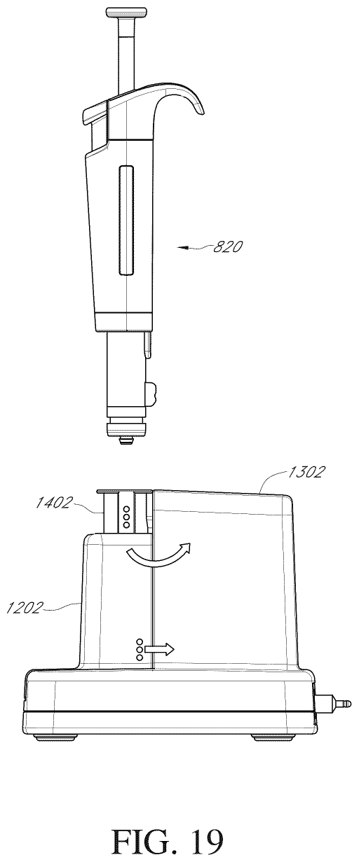

FIGS. 18A and 18B illustrate that, when the pipette station guard 1202 is connected to the pipette station 1302 and the reservoir 1402 is connected to the pipette station guard 1202 as discussed hereinabove, a pipette 820 and associated pipette tip 802 may be inserted into the reservoir 1402 to facilitate electroporation. FIG. 19 shows that, as noted above, the directions for withdrawing or inserting the pipette 820 (and/or associated pipette tip) from the reservoir 1402 are different from the directions in which the locking hooks of the pipette station guard 1202 are configured to retract from engagement with the hook catches of the pipette station 1302. Similarly, FIG. 19 indicates that the directions for withdrawing or inserting the reservoir 1402 from the pipette station guard 1202 are different from the directions in which the locking hooks of the pipette station guard 1202 are configured to retract from engagement with the hook catches of the pipette station 1302. Such features may prevent inadvertent removal of parts from the pipette docking assembly during electroporation, thereby improving user safety.

Example Port Doors

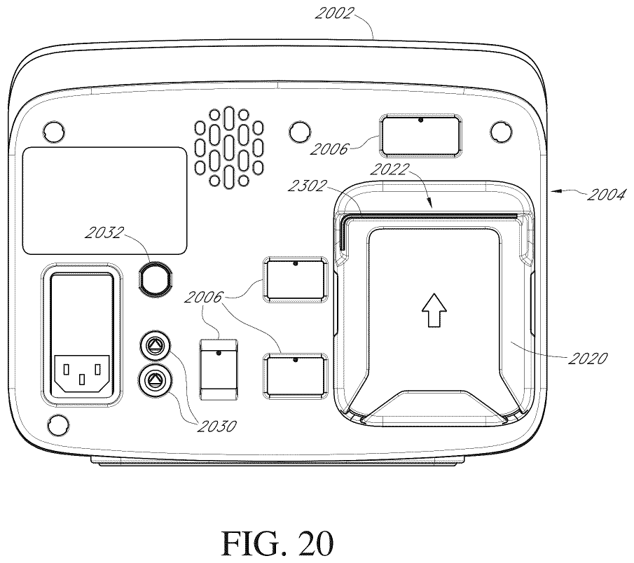

FIG. 20 depicts a rear panel 2004 of a pulse generator 2002 (e.g., corresponding to pulse generator 120) of an electroporation system. FIG. 20 shows that the pulse generator 2002 includes one or more connection ports (e.g., “USB PORT 1,” “USB PORT 2,” “LAN PORT,” “WIFI PORT,” etc.). In the example of FIG. 20, each connection port has a respective port door 2006. As noted above, the port doors 2006 may improve user safety (e.g., protecting users from high-voltage discharge through low-voltage components) and bring the pulse generator 2002 with applicable safety standards.

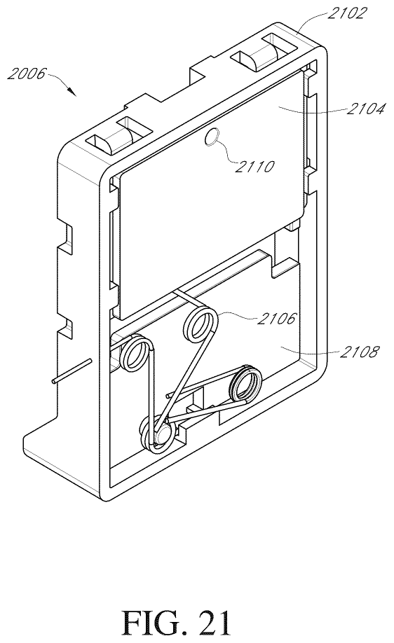

FIG. 21 provides an isolated view of an example port door 2006 of the pulse generator 2002. As shown in FIG. 21, the port door 2006 includes a door frame 2102 and a door 2104. The door 2104 is configured to translate vertically along the door frame 2102 to facilitate selective opening of the port door 2006 to selectively expose a connection port associated with the port door 2006 (though other configurations may be used, such as hinged doors). FIG. 21 also shows that the port door 2006 includes a biasing element 2106 that biases the door 2104 into a closed configuration for preventing access to the connection port associated with the port door 2006. Although FIG. 21 depicts the biasing element 2106 as a torsion spring, the biasing element 2106 may take on any suitable form.

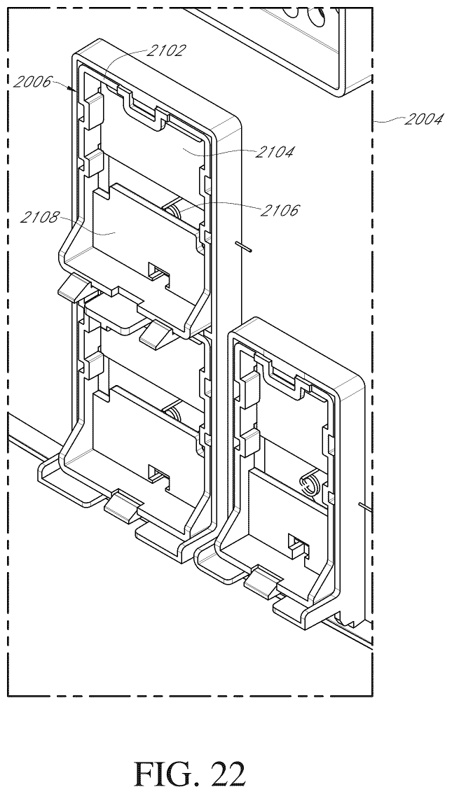

In the example of FIG. 21, the port door 2006 further includes a retention wall 2108 for retaining the biasing element 2106 between the retention wall 2108 and the rear panel 2004 of the pulse generator 2002 when the port door 2006 is connected to the rear panel 2004. FIG. 22 illustrates the port door 2006 snap-fit into connection with the rear panel 2004 (e.g., from an inner side of the rear panel 2004), with the biasing element 2106 arranged between the retention wall 2108 and the rear panel 2004.

FIG. 21 furthermore illustrates that the port door 2006 may include at least one tool interface 2110 on the door 2104 thereof. The tool interface 2110 may be configured to receive a tool (e.g., a hex tool, or any other type of tool) that a user may operate to facilitate selective opening of the door 2104 to access the connection port associated with the port door 2006 (e.g., by using the tool to overcome the biasing force of the biasing element 2106 to bring the door 2104 into an open configuration). One tool may be used for multiple port doors, and/or multiple tools may be associated with different port doors.

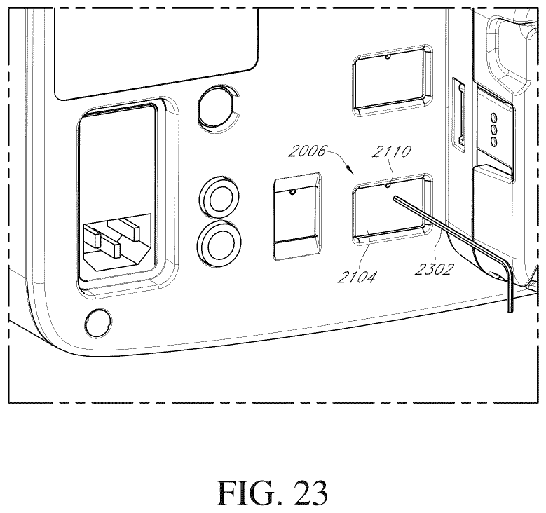

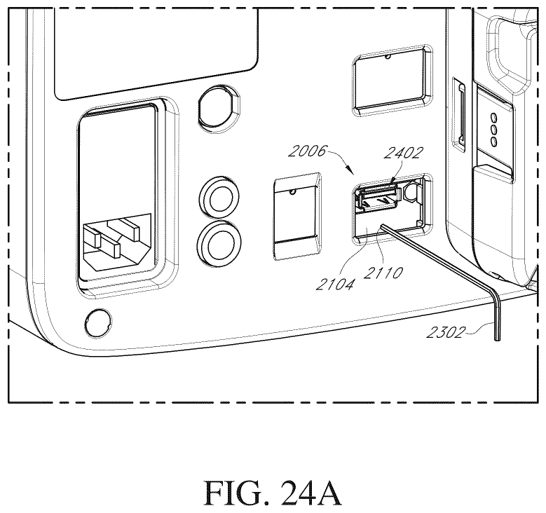

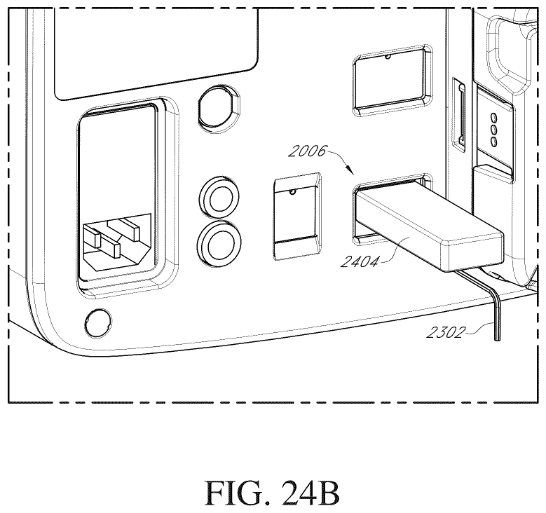

FIG. 23 shows a tool 2302 being inserted into the tool interface 2110 of the door 2104 of the port door 2006. With the tool 2302 inserted into the tool interface 2110, a user may apply force to the tool 2302 (e.g., downward force in the present example) to overcome the biasing force of the biasing element 2106 of the port door 2006. FIG. 24A shows the port door 2006 in an open configuration responsive to force applied to the door 2104 via the tool 2302 overcoming the biasing force of the biasing element 2106 of the port door 2006. As shown in FIG. 24A, the connection port 2402 associated with the port door 2006 is exposed when the port door 2006 is in the open configuration.

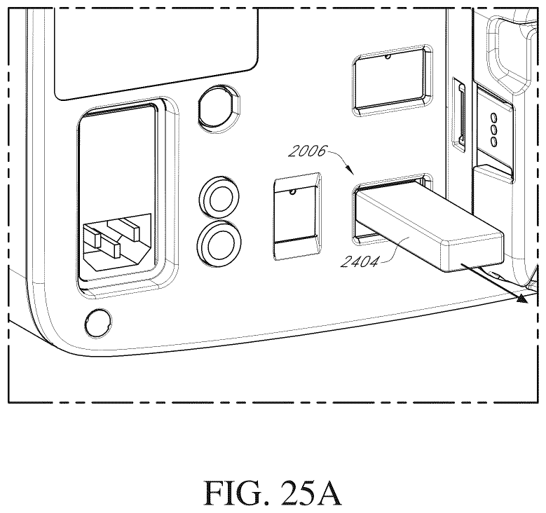

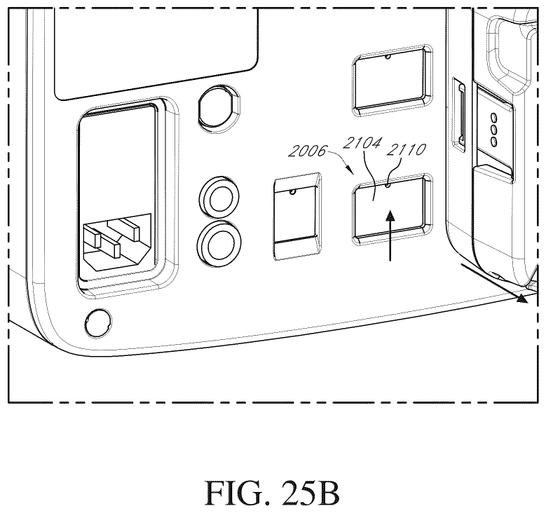

FIG. 24B illustrates a connection component 2404 (e.g., a USB drive) inserted into the connection port 2402 associated with the port door 2006. Insertion of the connection component 2404 into the connection port 2402 while the port door is in the open configuration allows the connection component 2404 to maintain the counteracting of the biasing force of the biasing element 2106 of the port door 2006, thereby allowing the port door 2006 to remain in the open configuration even after the tool 2302 is removed from the tool interface 2110. FIG. 25A shows the connection component 2404 maintaining the open configuration of the port door 2006 in the absence of the tool 2302. With the port door 2006 in the open configuration, the connection component 2404 may be removed from the connection port 2402 without intervention by the tool 2302 (as indicated by the bolded arrow in FIG. 25A). Disconnection of the connection component 2404 from the connection port 2402 removes the counteracting force on the biasing element 2106 of the port door 2006, allowing the biasing element 2106 to automatically force the port door 2006 back into the closed configuration (indicated by the bolded arrow in FIG. 25B) upon removal of the connection component 2404 and without additional user action to cause closing of the port door 2006.

In some implementations, the tool 2302 for facilitating selective opening of port doors 2006 of a pulse generator 2002 may be conveniently mountable on the pulse generator 2002 (and/or on another component of an electroporation system). For instance, FIG. 20 illustrates the tool 2302 mounted to a tool holder 2022 of the pulse generator 2002. In the example of FIG. 20, the tool 2302 is magnetically mounted to the tool holder 2022 arranged on a cable adapter 2020 of the pulse generator 2002. Additional details related to the cable adapter 2020 of the pulse generator 2002 will be provided hereinbelow.

Example Cable Components

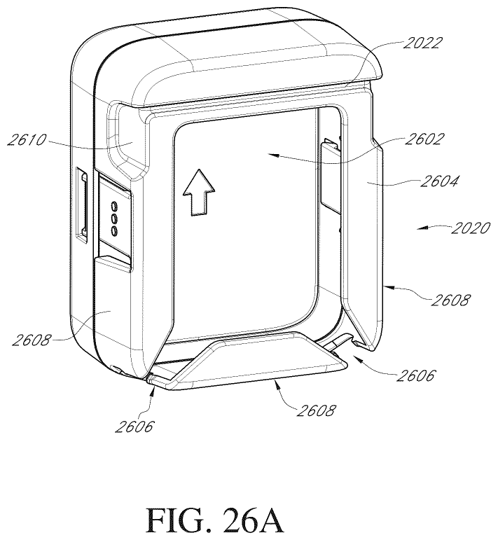

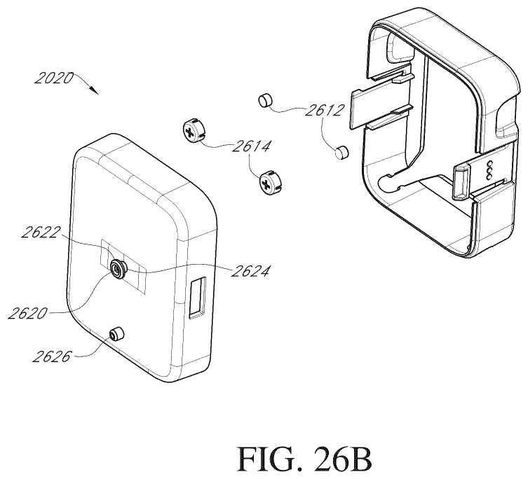

FIG. 26A provides an isolated view of the cable adapter 2020 of the pulse generator 2002. The cable adapter 2020 is configured to hold various cables that connect to the pulse generator 2002. For example (referring briefly to FIG. 20), the pulse generator 2002 may include high voltage connection ports 2030 and a low voltage connection port 2032. The high voltage connection ports 2030 may be configured to receive high voltage cables that may also connect to high voltage ports of a pipette station to facilitate electroporation. The low voltage connection port 2032 may be configured to receive a low voltage cable that connects to a low voltage port of the pipette station to facilitate various functions associated with carrying out electroporation (e.g., process monitoring/execution, data acquisition, sensor operation/monitoring, etc.).

In this way, the high voltage cables and the low voltage cables may be selectively detachable from the pulse generator 2002, allowing for cable replacement in the event of cable failure. In some instances, the high voltage connection ports 2030 are female connection ports, which can promote user safety by reducing the likelihood that a user inadvertently contact the electrodes of the high voltage connection ports 2030.

The cable adapter 2020 may selectively hold at least part of high voltage and/or low voltage cables that are configured to connect to the pulse generator 2002. Such functionality may be beneficial, for example, to allow the pulse generator 2002 and components associated therewith to be stored or placed on a workstation (e.g., inside a biosafety cabinet) in a space-efficient manner (e.g., especially where the pulse generator 2002 is configured to connect via cabling to multiple pipette stations and/or multiple external components).

In the example of FIG. 26A, the cable adapter 2020 includes an outer opening 2602 on (or at least partially defined by) a first surface 2604 (e.g., rear surface) of the cable adapter 2020. The outer opening 2602 exposes the interior of the cable adapter 2020 that can selectively hold at least a portion of high voltage and/or low voltage cables associated with the pulse generator 2002. The outer opening 2602 can allow user to selectively place, re-arrange, or remove high voltage and/or low voltage cables within the interior of the cable adapter 2020.

The cable adapter 2020 of FIG. 26A further includes one or more cable slots 2606 that extend from the outer opening 2602 and are at least partially arranged on (or are at least partially defined by) other surfaces 2608 of the cable adapter 2020. The cable slots 2606 also expose the interior of the cable adapter 2020 and may provide users with convenient cable entry points into and/or exit points from the interior cable adapter 2020 to enable effective cable management.

As noted above, the cable adapter 2020 of FIG. 26B includes the tool holder 2022 that can hold a port door tool 2302. The cable adapter 2020 may comprise one or more finger depressions 2610 to allow users to easily remove the tool 2302 from the tool holder 2022.

As further noted above, the tool holder 2022 may be configured to magnetically hold the tool 2302 (which may be magnetizable or magnetic). FIG. 26B illustrates magnets 2612 that may be arranged on the interior of the cable adapter 2020 (e.g., with magnet caps 2614 placed thereover) to enable the tool 2302 to be secured by the magnets 2612 when placed within the tool holder 2022 of the cable adapter 2020. One will appreciate, in view of the present disclosure, that other types of affixation methods for securing the tool 2302 within the tool holder 2022 are within the scope of the present disclosure.

As shown in FIG. 26B the cable adapter 2020 may include a mounting feature 2620 to facilitate selective mounting of the cable adapter 2020 to the pulse generator 2002. In the example of FIG. 26B, the mounting feature 2620 includes a disc 2622 positioned on a mounting arm 2624 extending from a surface of the cable adapter 2020. Other configurations and/or types of mounting features may be used. The disc 2622 may comprise a greater radius or transverse width/thickness than the mounting arm 2624 to facilitate engagement with a corresponding mounting hole of the pulse generator 2002. FIG. 26B also shows that the cable adapter 2020 may include additional features associated with mounting the cable adapter 2020 to the pulse generator 2002, such as an alignment feature 2626.

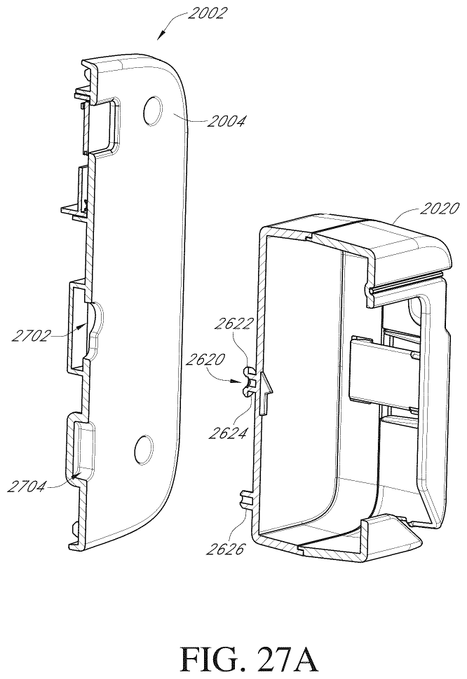

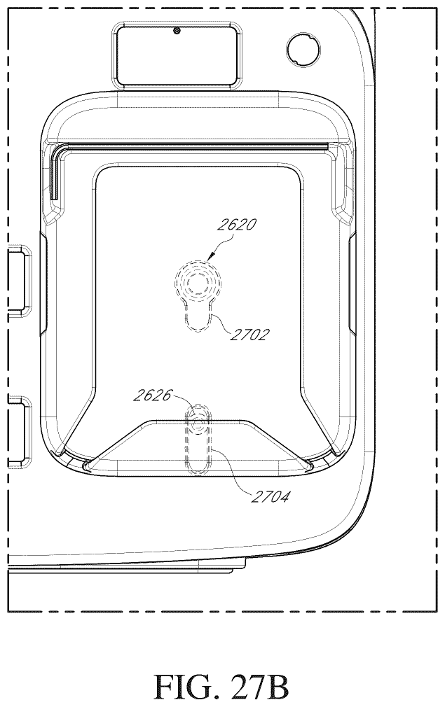

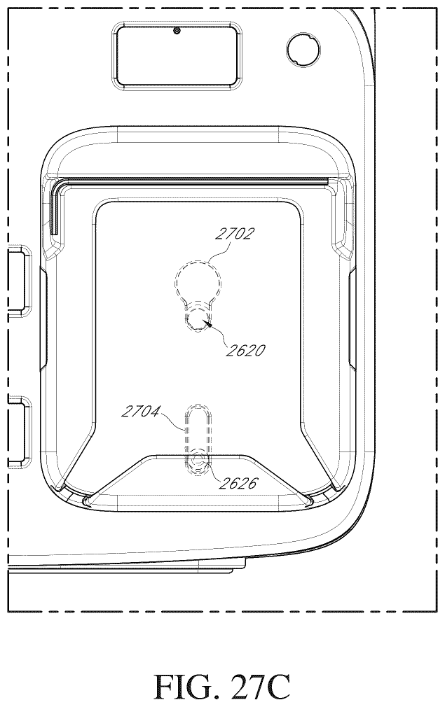

FIG. 27A shows a sectional view of the cable adapter 2020 and the pulse generator 2002. As illustrated, the pulse generator 2002 includes a mounting hole 2702 for receiving the mounting feature 2620 of the cable adapter 2020. In the example of FIG. 27, the mounting hole 2702 is defined by a pair of conjoined holes with a first (top) hole that has a larger radius than a second (bottom) hole arranged below the first hole. The radius of the first hole is greater than the radius of the disc 2622 of the mounting feature 2620 of the cable adapter 2020. The second hole has a smaller radius than the first hole and the radius of the disc 2622 of the mounting feature 2620 of the cable adapter 2020. The radius of the second hole is greater than the radius of the mounting arm 2624 of the mounting feature 2620 of the cable adapter 2020.

The disc 2622 is thus able to insert into the mounting hole 2702 through the first hole, as indicated by the indicated by the bolded arrow of FIG. 27A and as shown in FIG. 27B. After insertion therethrough, the cable adapter 2020 may be permitted to drop until the mounting arm 2624 of the mounting feature 2620 rests on the second hole of the mounting hole 2702, as indicated by the bolded arrow in FIG. 27C. The disc 2622 can then prevent removal of the cable adapter 2020 from the pulse generator 2002 until the cable adapter is lifted to bring the disc toward the first hole of the mounting hole 2702.

As shown in FIG. 27A, the alignment feature 2626 of the cable adapter 2020 may interface with a corresponding alignment feature 2704 of the pulse generator 2002 (implemented in the illustrated example as an elongated channel or slot). Such interfacing may impose a particular alignment (e.g., vertical alignment in the example shown) of the cable adapter 2020 with the pulse generator 2002 when the disc 2622 is inserted through the mounting hole 2702 and the alignment feature 2626 interfaces with the corresponding alignment feature 2704. The alignment feature 2704 may thus define a final position for the cable adapter 2020 relative to the pulse generator when the mounting feature 2620 of the cable adapter 2020 is mounted to the mounting hole 2702 of the pulse generator (thereby mitigating unintended displacement or rotation of the cable adapter 2020 when mounted to the pulse generator 2002).

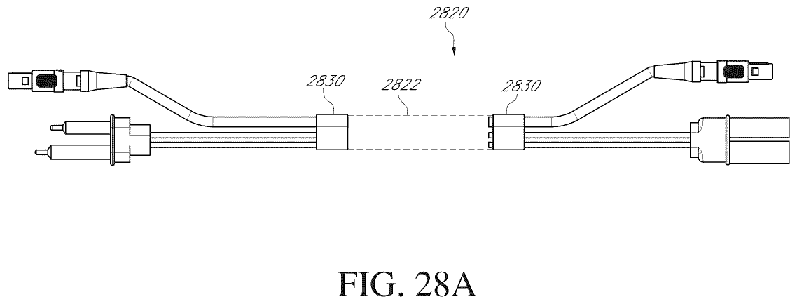

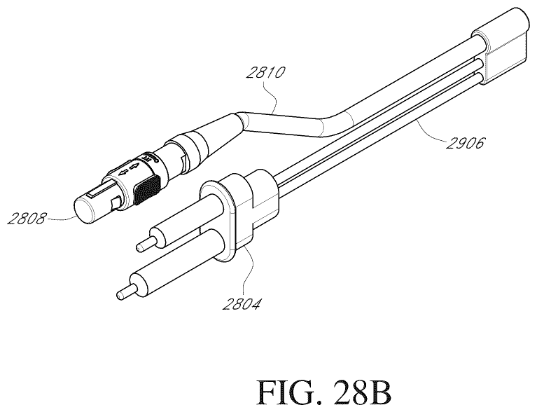

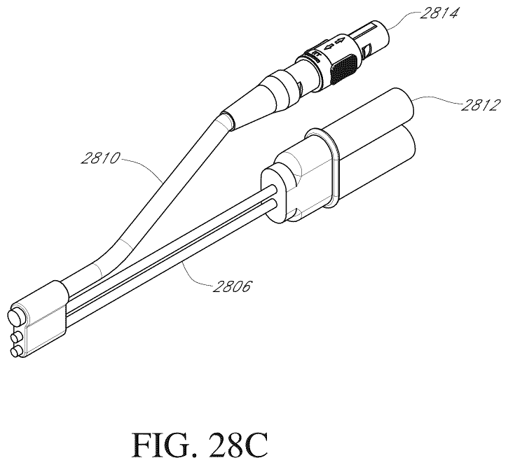

FIG. 28A illustrates an example cable assembly 2802 that may be used in an electroporation system. The cable assembly 2802 may comprise components for connecting to a pulse generator (labeled “PULSE GENERATOR” in FIG. 28A) and, at an opposing end, components for connecting to a pipette station (labeled “STATION” in FIG. 28A). FIG. 28B illustrates example aspects of the components for connecting to a pulse generator, which may include a high-voltage connector 2804 of high-voltage cables 2806 and a low-voltage connector 2808 of a low-voltage cable 2810. Similarly, FIG. 28C illustrates example aspects of components for connecting to a pipette station, which may include a corresponding high-voltage connector 2812 of the high-voltage cables 2806 and a corresponding low-voltage connector 2814 of the low-voltage cable 2810.

FIG. 28A also illustrates that the cables of the cable assembly 2802 (e.g., high-voltage cables 2806 and low-voltage cable 2810) may be bound or coupled to one another via a binding assembly 2820. In the example of FIG. 28A, the binding assembly 2820 includes a braided sleeve 2822 which surrounds at least a section of the high-voltage cables 2806 and the low-voltage cable 2810. The braided sleeve 2822 of FIG. 28A is connected to clips 2830, which also surround respective sections of the high-voltage cables 2806 and the low-voltage cable. The clips 2830 may be formed from any suitable material, such as a polymer material.

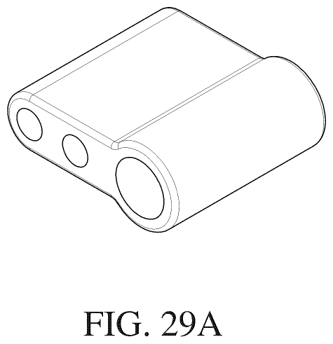

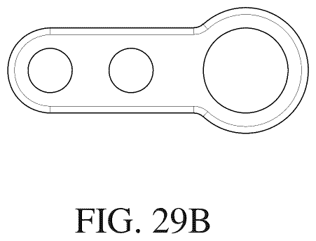

In some implementations, each of the clips 2830 is formed from multiple parts that are affixed to one another around the high-voltage cables 2806 and the low-voltage cable 2810. FIG. 29A illustrates a first part 2902 and a second part 2904 to form one of the clips 2830. When affixed to one another, as depicted in FIG. 29B, the first part 2902 and the second part 2904 form a plurality of conjoined holes 2906, which may surround the high-voltage cables 2806 and the low-voltage cable 2810. The first part 2902 and the second part 2904 may be combined in a variety of ways, such as by ultrasonic welding.

Example Aspects of Electrical Pulse Application

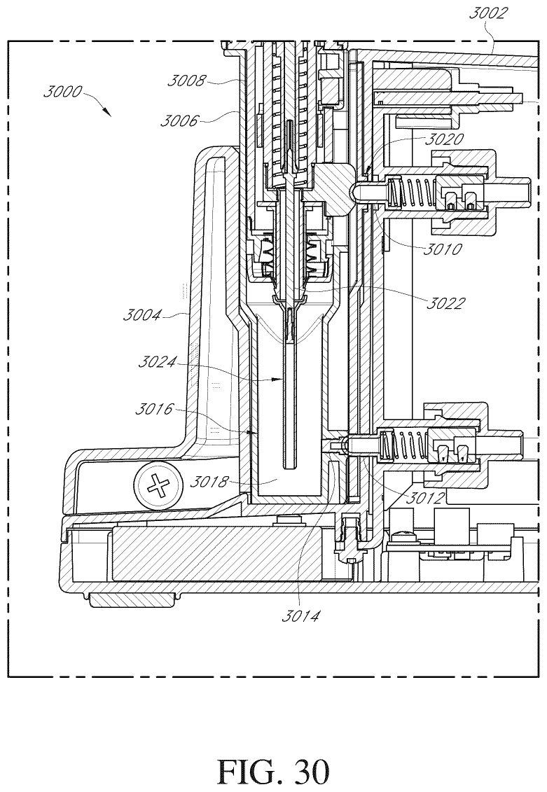

FIG. 30 illustrates a schematic representation of an electrical pulse applied using an electroporation system 3000 (e.g., corresponding to electroporation system 100) that includes a pipette station 3002 (e.g., corresponding to pipette station 1302), a pipette station guard 3004 (e.g., corresponding to pipette station guard 1202) mounted to the pipette station 3002, a buffer reservoir? 3006 (e.g., corresponding to buffer tube 1402) mounted to the pipette station guard 3004, and a pipette 3008 (e.g., corresponding to pipette 820 with pipette tip 802 attached thereto) inserted into the buffer tube 3006.

The pipette station 3002 of FIG. 30 includes electrodes 3010 and 3012 that may electrically connect to a pulse generator via high-voltage cables and/or ports as discussed hereinabove. The buffer tube 3006 also includes a buffer reservoir electrode 3014 (e.g., corresponding to electrode 1418) that is exposed to the interior of the buffer tube 3006 (e.g., the interior of a buffer reservoir 3016 of the buffer tube 3006) and is configured to contact the electrode 3012 of the pipette station 3002 when the buffer tube is mounted to the pipette station guard 3004 mounted to the pipette station 3002. The buffer reservoir electrode 3014 thus allows electrical pulses from the pulse generator to reach buffer solution 3018 within the buffer reservoir 3016 of the buffer tube 3006 (e.g., by completing a circuit for the electrical pulse to reach a sample within the pipette tip 3024 through the pipette 3008 and return through the buffer solution 3018 and buffer reservoir electrode 3014).

The buffer tube 3006 further comprises an electrode opening (corresponding to the electrode opening 1420 of FIG. 14) through which a pipette electrode 3020 of the pipette 3008 can extend when the pipette 3008 is mounted to the buffer tube 3006 (which is mounted to the pipette station guard 3004, which is mounted to the pipette station 3002) to allow the pipette electrode 3020 to contact the electrode 3010 of the pipette station 3002. The pipette electrode 3020 is in electrical communication with the plunger 3022 of the pipette tip 3024 associated with the pipette 3008, and the plunger 3022 is configured to contact with any sample disposed within the pipette tip 3024.

Thus, one or more high voltage pulses may be supplied by the pulse generator to a sample within the pipette tip 3024 to electroporate the sample when: (i) the pipette tip 3024 is connected to the pipette 3008 and disposed within the buffer solution 3018 to expose the sample within the pipette tip 3024 to the buffer solution 3018 at the tip opening of the pipette tip 3024, (ii) the buffer reservoir electrode 3014 is in contact with the buffer solution 3018 and the electrode 3012 of the pipette station 3002, (iii) the electrode 3010 of the pipette station 3002 is in contact with the pipette electrode 3020, and (iv) the pipette electrode 3020 is in electrical communication with the plunger 3022 of the pipette tip, which is contact with the sample in the pipette tip.

In some implementations, an electroporation system includes one or more sensors for determining whether the station guard 3004, the buffer tube 3006, and/or the pipette are properly positioned relative to one another to prevent high-voltage discharge unless the components are properly interconnected. Such sensors may take on any suitable form, and sensor data acquired thereby may be communicated via the low-voltage cable.

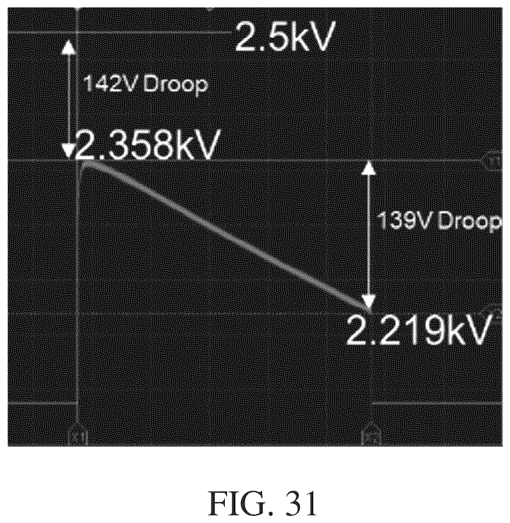

As noted above, conventional electroporation systems utilize an open loop pulse generator which uses a charging circuit to charge a large capacitor to the target voltage and then uses a high speed, high voltage electronic switch that connects the voltage at the capacitor to the pipette station. In some circumstances (e.g., when load resistance is small and/or for long pulse durations), the voltage that reaches the sample starts to droop through the duration of the pulse (e.g., because the charging electronics fail to provide enough power to sustain the load at the target voltage). Voltage droop can thus occur, as shown in FIG. 31 (for a target voltage of 2.5 kV).

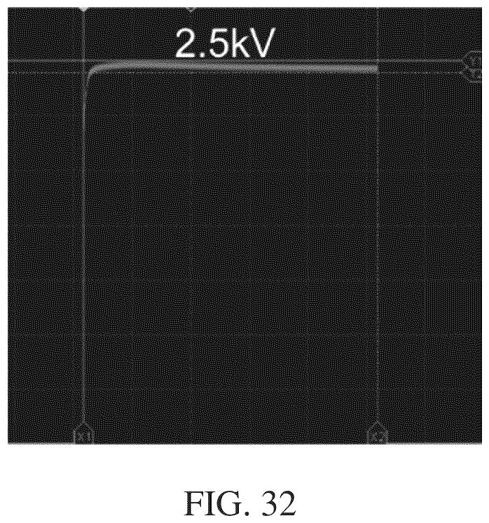

A pulse generator of the present disclosure (e.g., pulse generator 120) may utilize feedback loop control in which capacitors are charged to a voltage above the target voltage, and the voltage is regulated as required to produce a steady voltage supply at the desired voltage (and/or pulse width and/or modulation settings). FIG. 32 illustrates an example flat pulse waveform that can be applied utilizing a pulse generator according to the present disclosure.

In some implementations, the pulse generator comprises one or more voltage sources configured to charge one or more high-voltage capacitors. The high-voltage capacitor(s) is/are configured to operate as a power supply for an amplifier circuit. the amplifier circuit is configured to supply voltage to a sample associated with a pipette in a manner that accounts for variations in load. For example, load can vary for different reaction conditions (e.g., buffer solution type, pipette tip size, cell concentration, etc.) and/or throughout electroporation processes (e.g., based on changes in temperature).

The amplifier circuit may include a common source amplifier configured to output a high-voltage pulse. The common source amplifier may receive a signal from an amplitude setting loop. The signal of the amplitude setting loop is based upon input from a digital-to-analog converter and input from a voltage sensing loop. The input from the digital-to-analog converter may correspond to a user-selected waveform (e.g., sine wave, triangle wave, square wave, etc.). The input from the voltage sensing loop is determined using the high-voltage pulse, a voltage divider, and a differential amplifier. The common source amplifier may amplify the signal from the amplitude setting loop by a factor of about 1,000 to about 2,000 (e.g., 1,250).

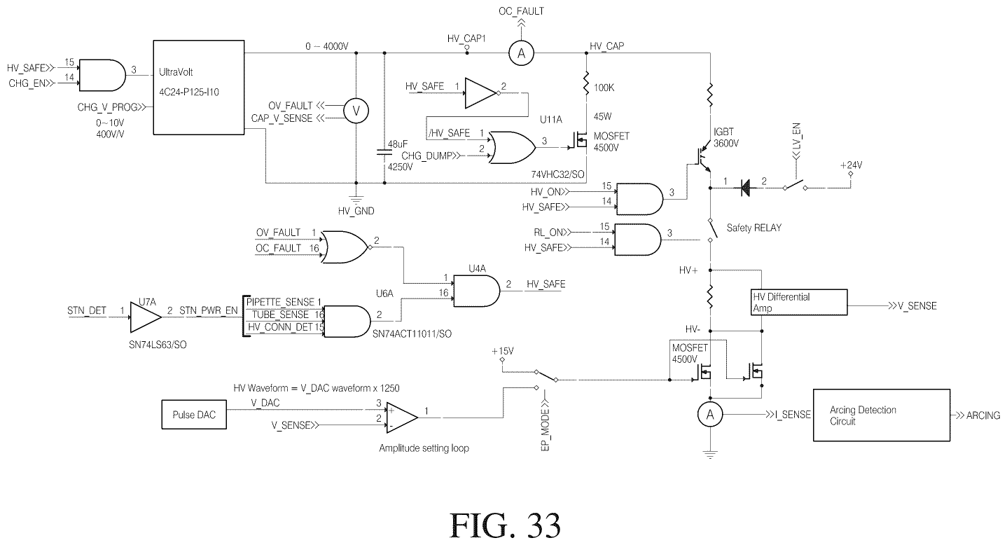

FIG. 33 illustrates an example architecture that may be implemented in a pulse generator of an electroporation system, in accordance with implementations of the present disclosure. As illustrated, the circuit design for the pulse generation converts the HV voltage to V_SENSE (e.g., HV_Voltage/1250) using a voltage divider and a differential amplifier (e.g., a “voltage sensing loop”). The pulse waveform to the V_DAC output from DAC (digital-to-analog converter) is compared with V_SENSE and amplified to facilitate low side switching of the high voltage MOSFET (e.g., an “amplitude setting loop”). Based upon the amplitude of the high voltage output at HV_Voltage, the V_DAC amplitude is set to HV_Voltage/1250, and the waveform at V_DAC sets the waveform at the high voltage output at port HV+.

A pulse generator architecture as presently disclosed may enable waveform customization, allowing implementation of sine waves, square waves, triangle waves, sawtooth waves, and/or other waveforms.

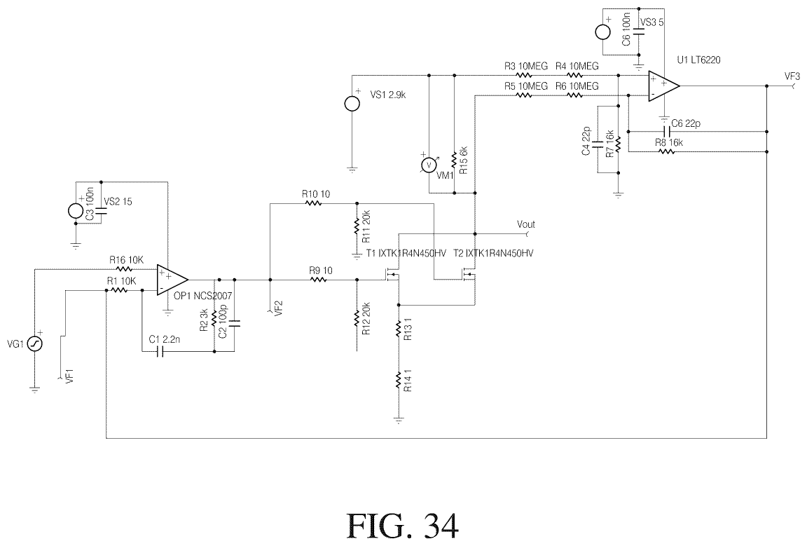

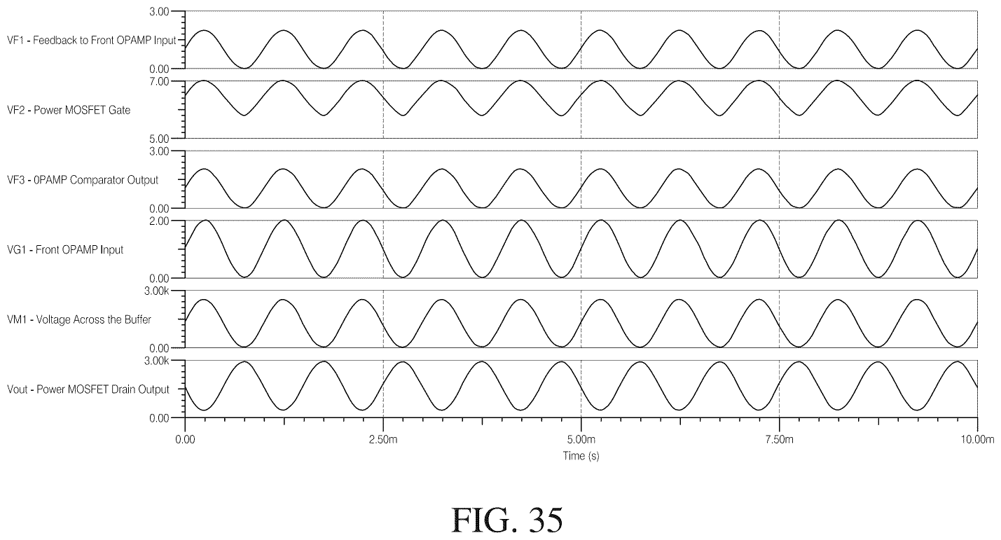

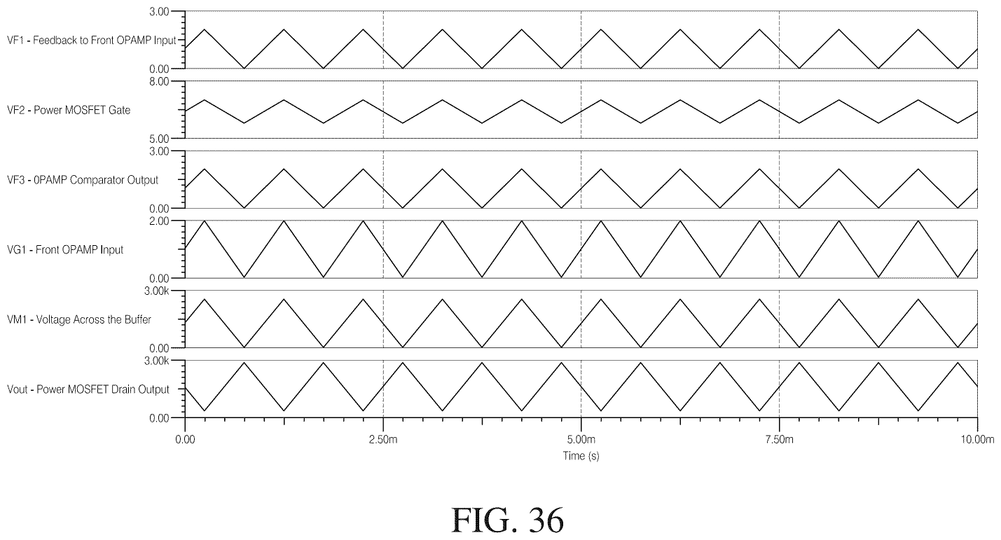

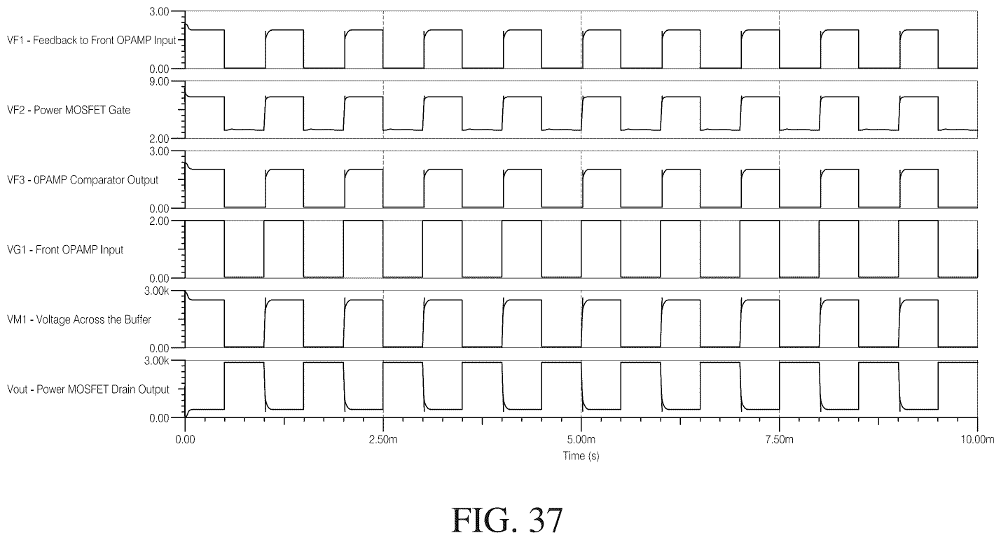

FIG. 34 illustrates a simulation-based integrated circuit (SPICE model or Simulation Program with Integrated Circuit Emphasis model) that optimizes the component values (e.g., resistors, capacitors). The illustrated circuit is designed to generate a flat pulse. FIGS. 35 through 37 illustrate example waveform simulations of different simulated waveforms implementing the disclosed architecture. FIG. 35 illustrates an example sinewave with an input (e.g., VG1) of 1 kHz frequency, 1 V amplitude, and 1 V offset, and the corresponding output (e.g., VM1) of 1 kHz frequency, 1250 V amplitude, and 1250 V offset. FIG. 36 illustrates an example triangle with an input (e.g., VG1) of 1 kHz frequency, 1 V amplitude, and 1 V offset, and the corresponding output (e.g., VM1) of 1 kHz frequency, 1250 V amplitude, and 1250 V offset. FIG. 37 illustrates an example sinewave with an input (e.g., VG1) of 1 kHz frequency, 1 V amplitude, and 1 V offset, and the corresponding output (e.g., VM1) of 1 kHz frequency, 1250 V amplitude, and 1250 V offset.

Example Arcing Detection Modules

As noted above, during high voltage electroporation (500V to 2500V) arcing may occur if any bubbles are introduced into the tip holding the cell sample. Arcing can cause electroporation failure and/or poor transfection results. Human observation has been traditionally relied upon to detect arcing, which can lead to detection errors and/or failures. Accordingly, at least some disclosed embodiments implement an arcing detection module configured to detect arcing without relying on human observation.

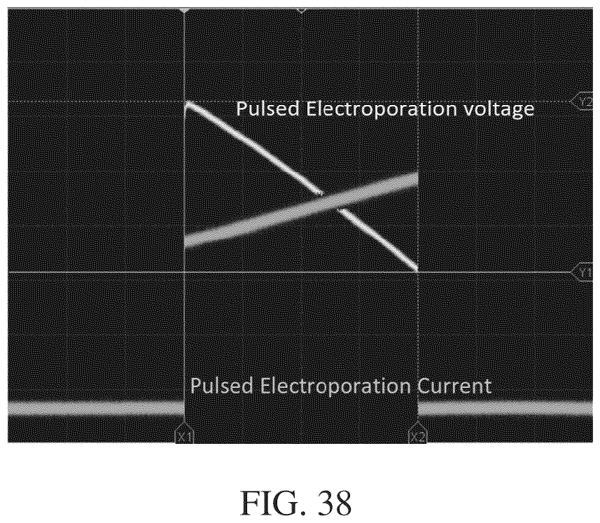

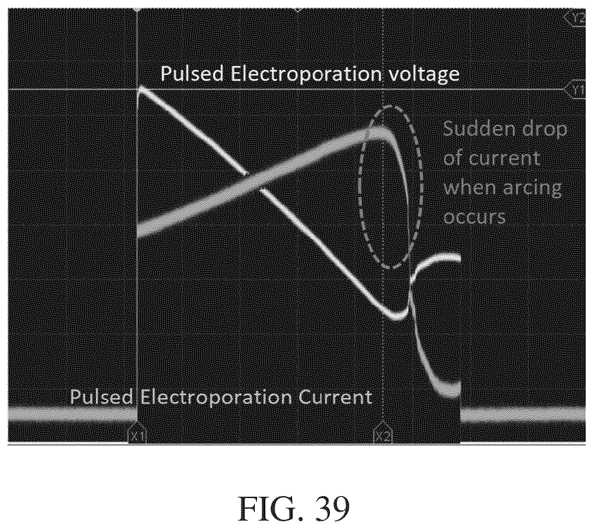

Detection of a sudden drop in electroporation pulse current may be indicative of arcing. FIG. 38 illustrates an example voltage and current waveform for an electroporation pulse where no arcing occurs. FIG. 39 illustrates an example voltage and current waveform for an electroporation pulse that shows a sudden drop of current associated with arcing occurring.

Because different reaction conditions may exist, it is beneficial for the arcing detection module to be able to detect a current drop for different types of tips, buffers, load resistances (which can change throughout electroporation processes based on temperature), etc. An arcing detection module configured to detect arcing during electroporation may thus comprise (i) a first stage amplifier configured to provide an amplified current signal based on a current signal associated with the voltage applied to the sample (amplification of the current signal by the first stage amplifier may be based upon output of low voltage detection circuitry for determining resistance associated with the sample), (ii) a bandpass filter configured to filter a falling edge signal from the amplified current signal (where the falling edge signal is indicative of a drop in current passing through the sample, which is suggestive of arcing), and (iii) a comparator configured to compare the falling edge signal filtered by the bandpass filter to one or more reference criteria to determine whether arcing has occurred in the sample.

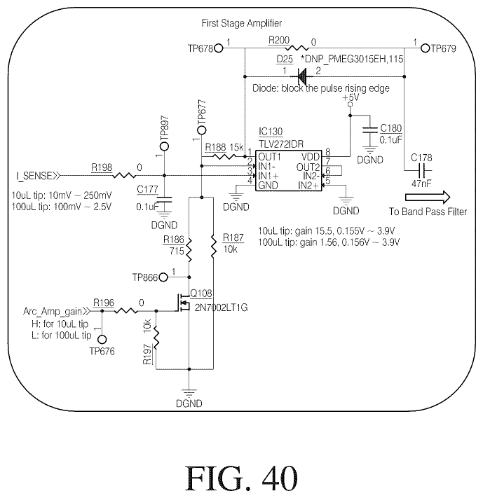

FIG. 40 illustrates an example first stage amplifier of an arcing detection module for implementation in an electroporation system. As illustrated, the pulsed electroporation current (“I_SENSE”) is sensed by a resistor (e.g., a 3 Ohm resistor) and converted to a voltage. A microcontroller unit (MCU) may provide the H/L signal (“Arc_Amp_gain”) based on the type of pipette tip (e.g., as determined based upon sensor data or user input, etc.) to adjust the gain of the first stage amplifier (e.g., the gain may be 10× when a 10 μL pipette tip is used relative to the gain used for a 100 μL pipette tip).

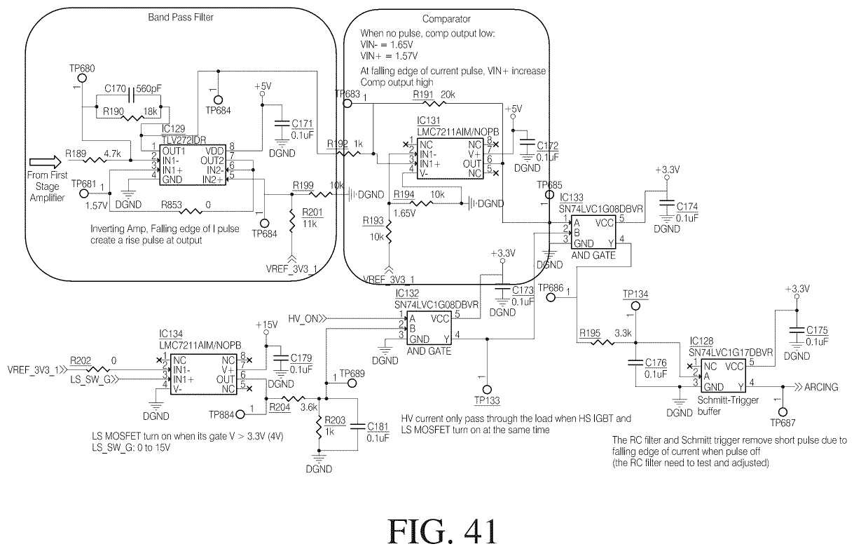

FIG. 41 illustrates an example band pass filter and comparator of the arcing detection module of FIG. 40. The first stage amplifier of FIG. 40 may connect to the band pass filter of FIG. 41, as indicated by the bolded arrow labeled “To Band Pass Filter” in FIG. 40 and by the bolded arrow labeled “From First Stage Amplifier” in FIG. 41.

The band pass filter may remove the normal current waveform and output the sudden current falling signal (if present) to the comparator. The comparator circuit may then determine if the current failing signal corresponds to an arcing event (e.g., based on comparison to a reference signal/reference data). The output of the arcing detection circuit may comprise a logic signal (“ARCING”), which connects to a microprocessor unit (MCU) as an input to indicate whether arcing is detected.

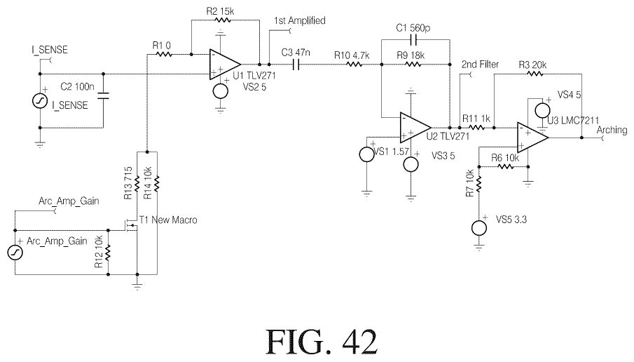

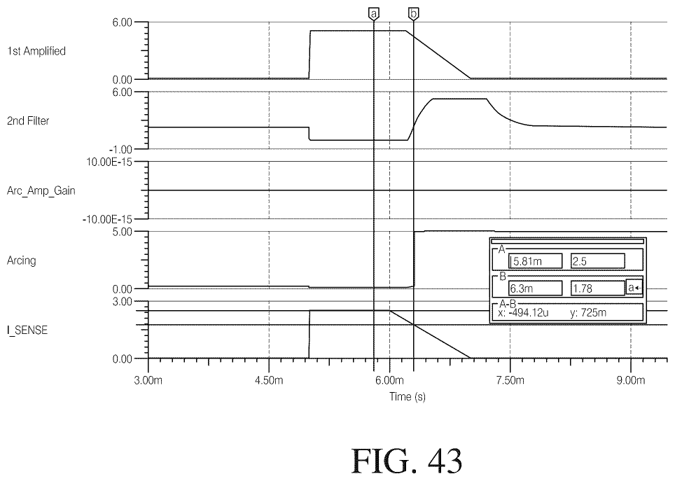

FIG. 42 illustrates a simulation-based integrated circuit (SPICE model) that optimizes the component values (e.g., resistors, capacitors, etc.). The illustrated circuit is designed to detect arcing with high sensitivity while avoiding misdetection. The values of the components can be chosen based on part availability. The input of the current profile is simplified as a sudden drop within 1 millisecond. Different starting current (e.g., from which the current begins to drop) are set to simulate different buffer types and different tip types to be used. FIG. 43 illustrates simulation results showing voltage (I_SENSE) dropping from 2.5V to 1.78V when arcing occurs and the arcing signal output correspondingly increasing. The arcing signal can be sent to the MCU as discussed above.

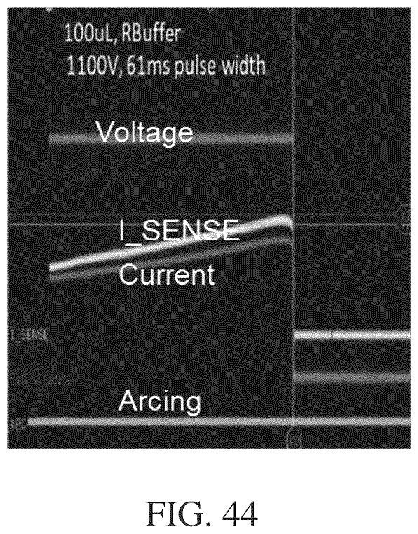

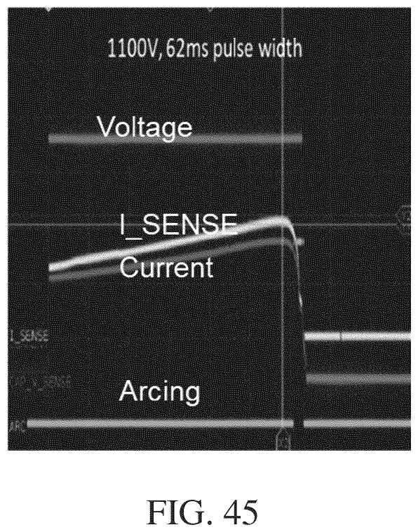

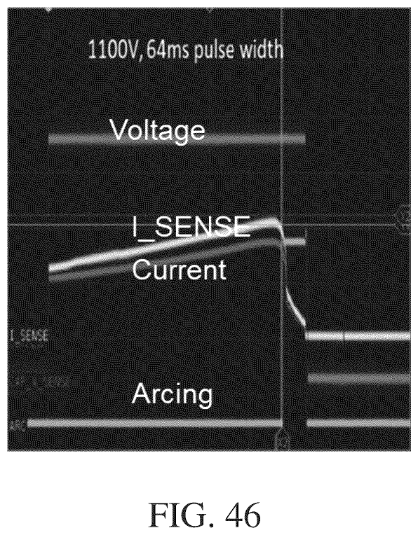

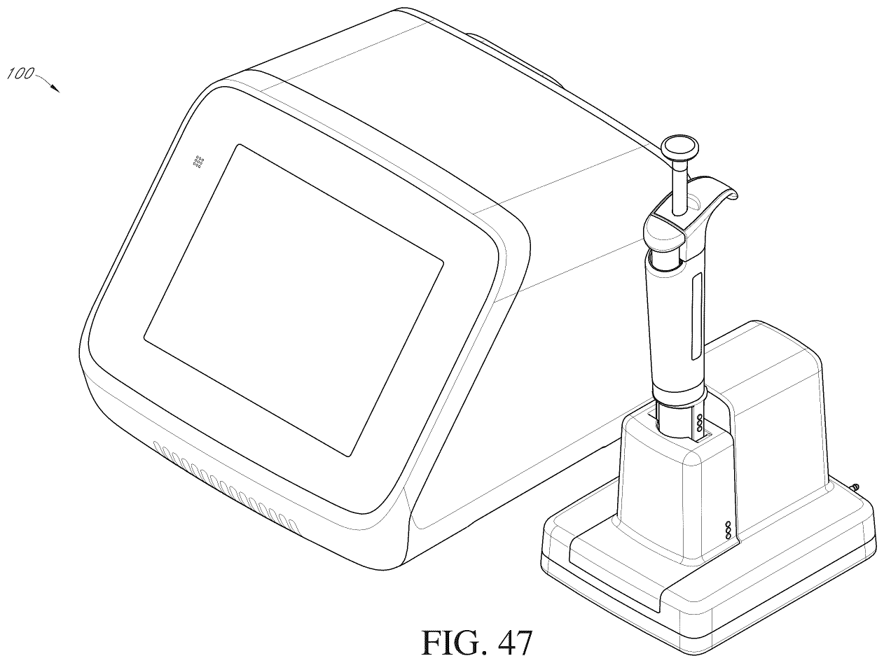

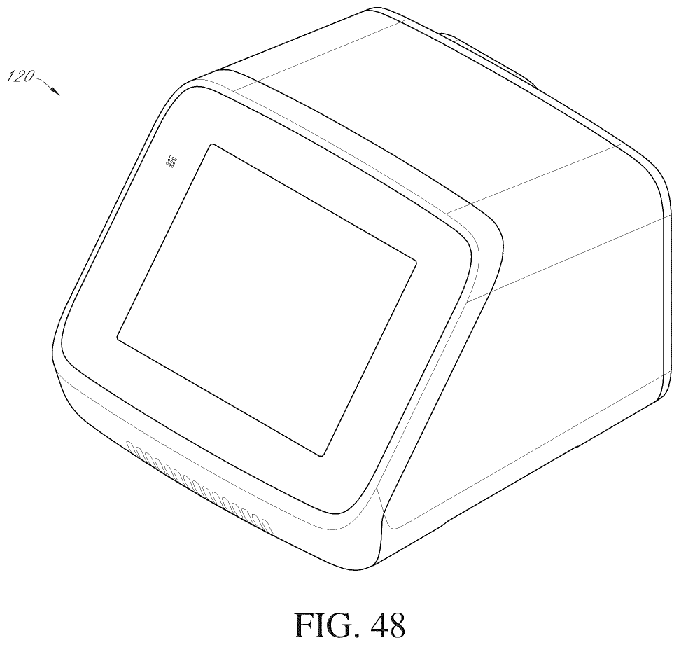

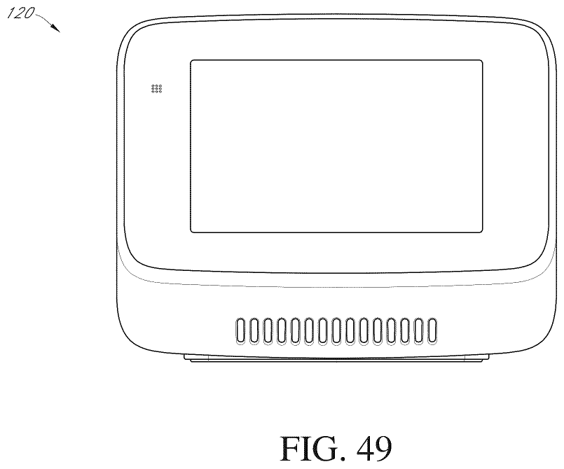

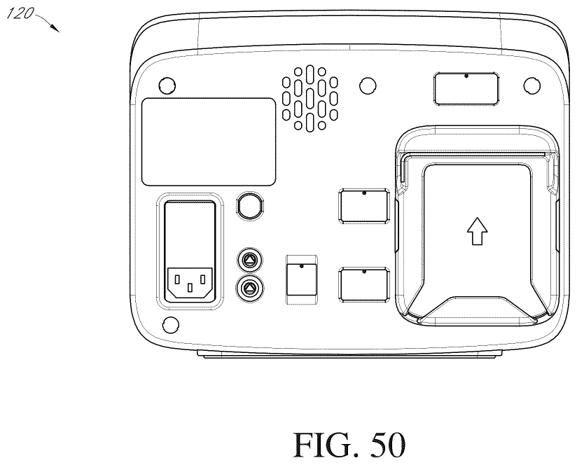

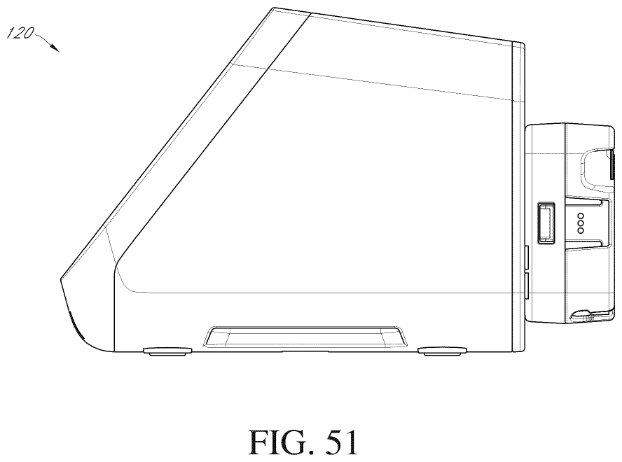

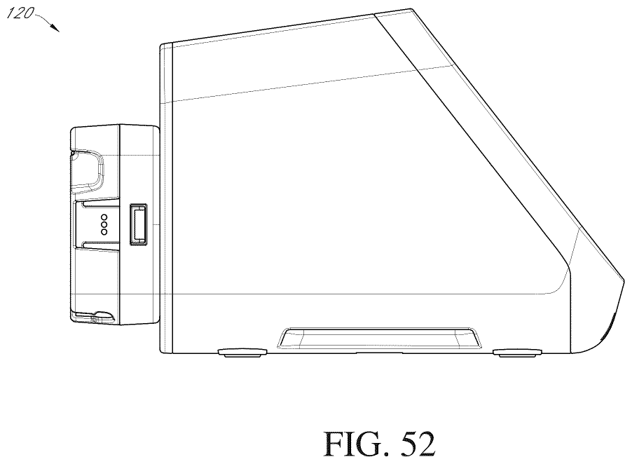

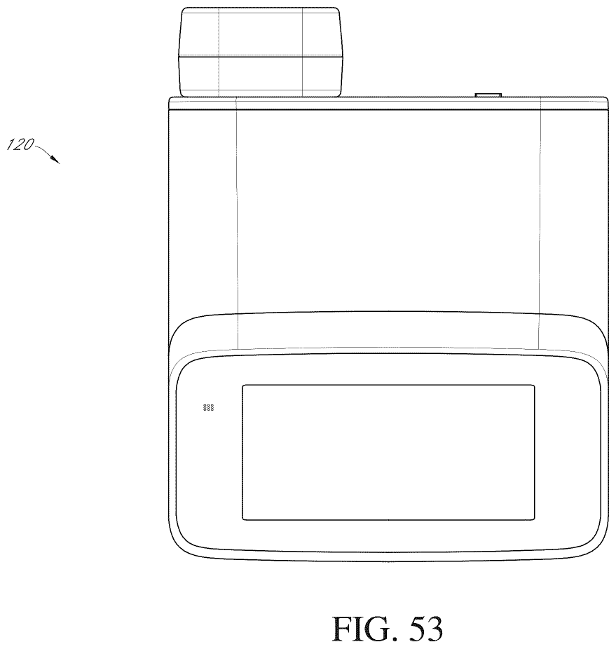

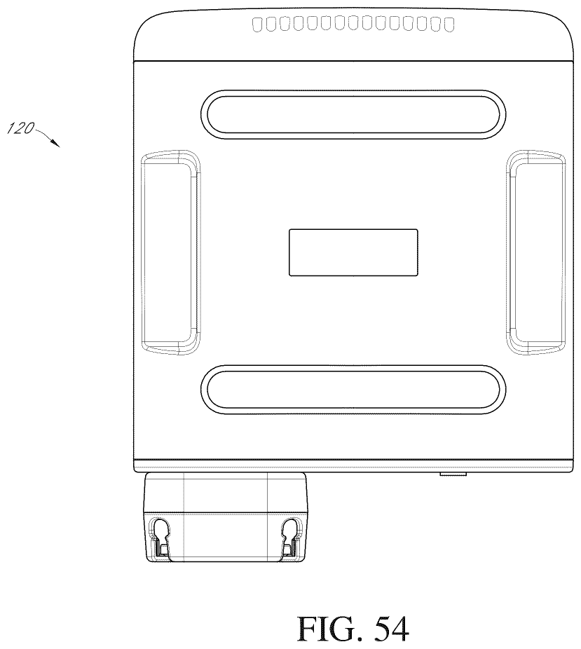

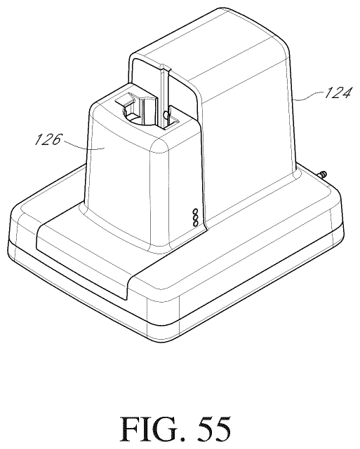

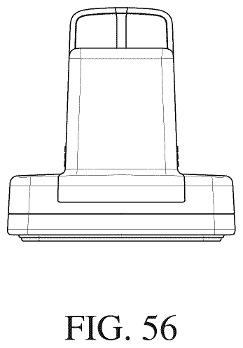

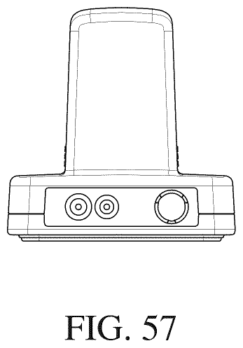

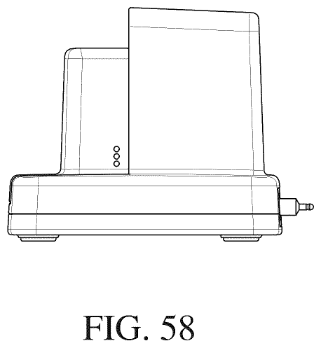

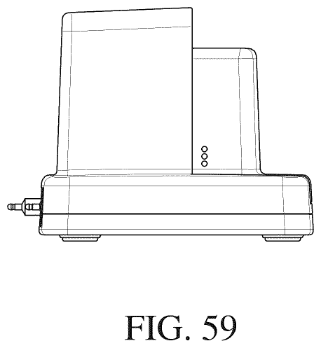

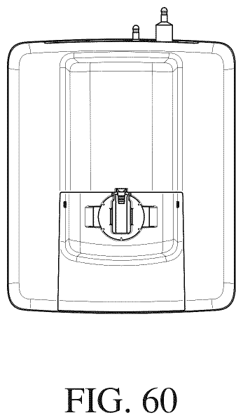

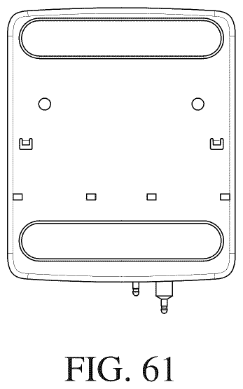

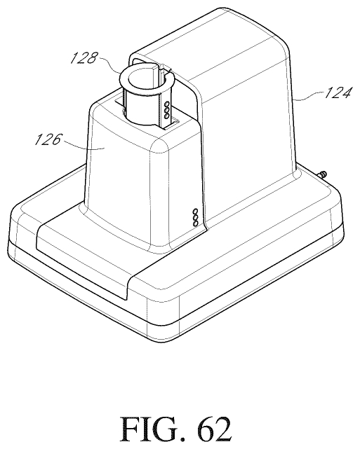

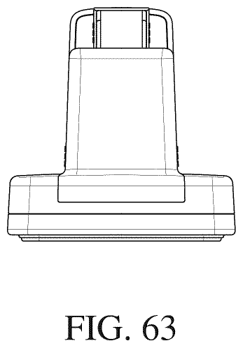

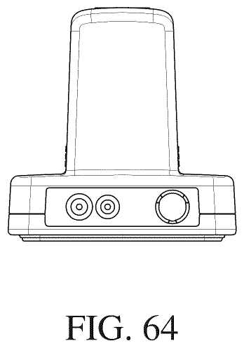

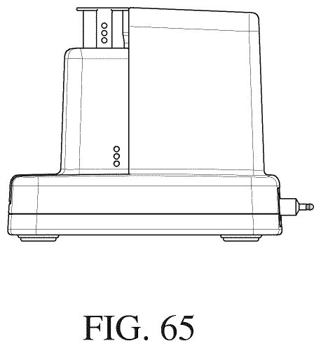

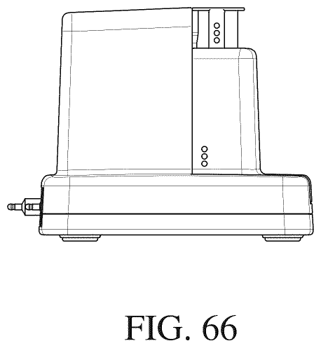

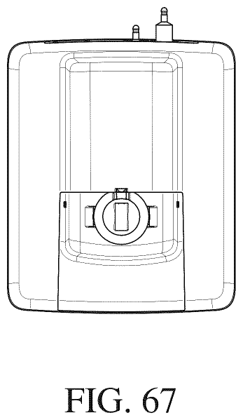

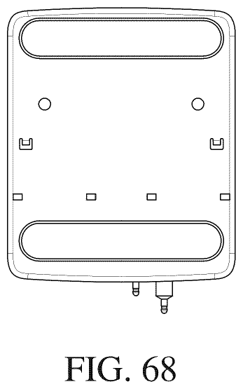

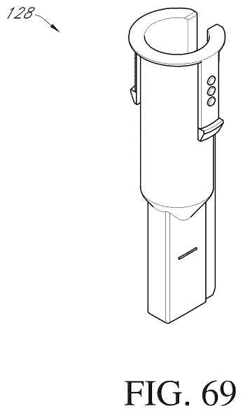

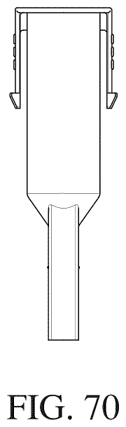

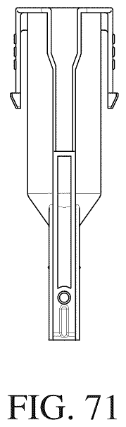

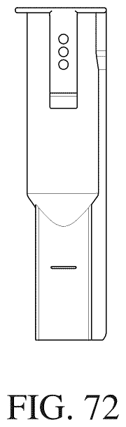

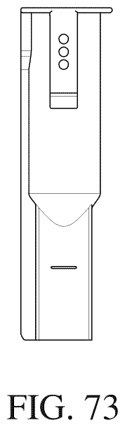

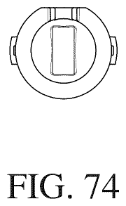

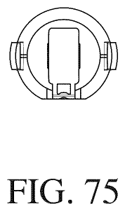

FIGS. 44 through 46 illustrate test results from tests performed to verify operation of the arc detection circuitry discussed above. The tests were performed under different voltage settings from 2,500 V to 500 V with different pulse widths and various numbers of pulses. The test results indicate a robust ARCING signal when a sudden drop in current occurs due to arcing during electroporation.

Additional Computer System Details

Disclosed embodiments may comprise or utilize a special purpose or general-purpose computer including computer hardware, as discussed in greater detail below. Disclosed embodiments also include physical and other computer-readable media for carrying or storing computer-executable instructions and/or data structures. Such computer-readable media can be any available media that can be accessed by a general-purpose or special-purpose computer system. Computer-readable media that store computer-executable instructions in the form of data are one or more “physical computer storage media” or “hardware storage device(s).” Computer-readable media that merely carry computer-executable instructions without storing the computer-executable instructions are “transmission media.” Thus, by way of example and not limitation, the current embodiments can comprise at least two distinctly different kinds of computer-readable media: computer storage media and transmission media.

Computer storage media (aka “hardware storage device”) are computer-readable hardware storage devices, such as RAM, ROM, EEPROM, CD-ROM, solid state drives (“SSD”) that are based on RAM, Flash memory, phase-change memory (“PCM”), or other types of memory, or other optical disk storage, magnetic disk storage or other magnetic storage devices, or any other medium that can be used to store desired program code means in hardware in the form of computer-executable instructions, data, or data structures and that can be accessed by a general-purpose or special-purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmission media can include a network and/or data links which can be used to carry program code in the form of computer-executable instructions or data structures, and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general-purpose computer, special purpose computer, or special purpose processing device to perform a certain function or group of functions. The computer-executable instructions may be, for example, binaries, intermediate format instructions such as assembly language, or even source code.

Disclosed embodiments may comprise or utilize cloud computing. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, wearable devices, and the like. The invention may also be practiced in distributed system environments where multiple computer systems (e.g., local and remote systems), which are linked through a network (either by hardwired data links, wireless data links, or by a combination of hardwired and wireless data links), perform tasks. In a distributed system environment, program modules may be located in local and/or remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Application-specific Standard Products (ASSPs), System-on-a-chip systems (SOCs), Complex Programmable Logic Devices (CPLDs), central processing units (CPUs), graphics processing units (GPUs), and/or others.

As used herein, the terms “executable module,” “executable component,” “component,” “module,” or “engine” can refer to hardware processing units or to software objects, routines, or methods that may be executed on one or more computer systems. The different components, modules, engines, and services described herein may be implemented as objects or processors that execute on one or more computer systems (e.g., as separate threads).

In some implementations, systems of the present disclosure may comprise or be configurable to execute any combination of software and/or hardware components that are operable to facilitate processing using machine learning models or other artificial intelligence-based structures/architectures. For example, one or more processors may comprise and/or utilize hardware components and/or computer-executable instructions operable to carry out function blocks and/or processing layers configured in the form of, by way of non-limiting example, single-layer neural networks, feed forward neural networks, radial basis function networks, deep feed-forward networks, recurrent neural networks, long-short term memory (LSTM) networks, gated recurrent units, autoencoder neural networks, variational autoencoders, denoising autoencoders, sparse autoencoders, Markov chains, Hopfield neural networks, Boltzmann machine networks, restricted Boltzmann machine networks, deep belief networks, deep convolutional networks (or convolutional neural networks), deconvolutional neural networks, deep convolutional inverse graphics networks, generative adversarial networks, liquid state machines, extreme learning machines, echo state networks, deep residual networks, Kohonen networks, support vector machines, neural Turing machines, and/or others.

It will also be appreciated that systems, processes, and/or products according to certain embodiments of the present disclosure may include, incorporate, or otherwise include properties features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, processes, products, and the like are not described herein in particular detail in order to avoid obscuring aspects of the example embodiments. Such aspects are, however, also contemplated herein.

While the instant disclosure provides certain illustrative aspects and describes the general principles of the described technology, those persons of ordinary skill in the relevant arts will appreciate that modifications in the arrangement and details of the disclosure may be introduced without departing from these aspects and principles. Accordingly, Applicant claims all modifications that are within the spirit and scope of the appended claims.

1. A pipette comprising:
a proximal section having a handle;
a distal section configured to reversibly attach to a pipette tip;
a first actuator disposed in the proximal section that when actuated is operable to control: i) a pipetting function of the pipette; and ii) grasping and ungrasping of a plunger disposed within a lumen of the pipette tip; and
a second actuator disposed in the proximal section that when actuated is operable to cause the pipette tip to detach from the distal section of the pipette.

2. The pipette of claim 1, further comprising a pipette electrode disposed in the distal section and electrically coupled to the plunger when grasped.

3. The pipette of claim 1, wherein the pipetting function comprises aspirating a fluid into, or dispensing a fluid from, a pipette tip attached to the distal section.

4. The pipette of claim 3, wherein the first actuator has a first undepressed position and a second partially depressed position, and wherein transitioning the first actuator from the first undepressed position to the second partially depressed position causes dispensing from the pipette tip and transitioning of the first actuator from the second partially depressed position to the first undepressed position causes aspirating into the pipette tip.

5. The pipette of claim 4, wherein the first actuator has a third fully depressed position.

6. The pipette of claim 5, wherein transitioning the first actuator from the second partially depressed position to the third fully depressed position causes ungrasping of the plunger, and transitioning the first actuator from the third fully depressed position to the second partially depressed position causes grasping of the plunger.

7. The pipette of claim 6, further comprising a gripper mechanism disposed in the distal section, the gripper mechanism operable to transition between a closed configuration and an open configuration upon transitioning of the first actuator between the second partially depressed position and the third fully depressed position.

8. The pipette of claim 7, wherein the gripper mechanism comprises:
a gripper jaw, the gripper jaw comprising a jaw opening for receiving an engagement section of the plunger; and
a gripping sleeve positioned around the gripper jaw configured to exert an inward force on the gripper jaw to cause the gripper jaw to exert a compressive force on the engagement section of the plunger to retain the engagement section of the plunger within the jaw opening when the first actuator is in the first depressed position or the second partially depressed position.

9. The pipette of claim 8, wherein the gripper jaw and gripping sleeve are configured to translate within the pipette while retaining the engagement section of the plunger within the jaw opening to cause translation of a lumen section of the plunger within the lumen of the pipette tip to facilitate the pipetting function.

10. The pipette of claim 9, wherein the gripper jaw is moved distally relative to the gripping sleeve when the first actuator is transitioned from the second partially undepressed position to the third fully depressed position thereby releasing the plunger from the gripper jaw.

11. The pipette of claim 10, the second actuator has a first undepressed position and a second depressed position.

12. The pipette of claim 11, further comprising a tip interface disposed circumferentially about the gripper jaw, the tip interface including a retention platform configured to engage tabs of an attachment interface of the pipette tip to secure a tip sleeve defining the lumen of the pipette tip to the distal section of the pipette.

13. The pipette of claim 12, further comprising a tip ejection sleeve operably connected to the second actuator and disposed adjacent to the tip interface, the tip ejection sleeve operable to move distally with respect to the tip interface when the second actuator is actuated by transitioning the second actuator from the first undepressed position to the second depressed position and displace the attachment interface of the pipette tip from the retention platform to detach the tip sleeve from the pipette.

14. The pipette of claim 13, wherein the second actuator is configured to traverse a blank travel distance when pressed prior to causing detachment of the one or more tabs from the retention platform.

15. The pipette of claim 14, wherein the gripper mechanism is configured to retain the engagement section of the plunger within the gripper opening throughout detachment of the attachment interface from the retention platform.

16. The pipette of claim 15, wherein, when the attachment interface and tip sleeve of the pipette tip are detached from the distal section of the pipette, transitioning the first actuator from the second partially depressed position to the third fully depressed position causes release of an engagement section of a plunger retained with the gripper jaw thereby releasing the pipette tip from the distal section of the pipette.

17. The pipette of claim 16, wherein the pipette electrode is electrically coupled to the gripper jaw of the gripper mechanism.

18. The pipette of claim 17, wherein the gripper jaw is composed of an electrically conductive material and operable to allow an electrical pulse applied to the pipette electrode to pass through the pipette electrode, through the gripper jaw, through the plunger retained within the jaw opening of the gripper jaw, through a sample containing cells contained within the lumen of the pipette tip, and through a second electrode disposed adjacent a distal end of the pipette tip, thereby electroporating the cells contained in the sample.

19-20. (canceled)

21. An electroporation system comprising:
a pipette of claim 1;
a pipette tip;
a pipette docking assembly; and
a pulse generator.

22-78. (canceled)

79. A pipette tip comprising:
a tip sleeve defining a lumen extending from a proximal end of the pipette tip to a distal end of the pipette tip;
a plunger at least partially disposed within the lumen, the plunger being composed of an electrically conductive material and configured to translate along the lumen to facilitate aspirating fluid into, and/or dispensing fluid from, the lumen; and
an attachment interface disposed at the proximal end of the pipette tip, the attachment interface comprising one or more tabs configured to engage with a distal section of a pipette.

80-132. (canceled)
